(19) United States
(12) Patent Application Publication
(10) Pub. No.: US 2023/0137672 A1
(43) Pub. Date: May 4, 2023

(54) IMMUNOTHERAPEUTIC TARGETS IN MULTIPLE MYELOMA AND METHODS FOR THEIR IDENTIFICATION

(71) Applicant: THE TRUSTEES OF INDIANA UNIVERSITY, Bloomington, IN (US)

(72) Inventor: Fabiana PERNA, Indianapolis, IN (US)

(21) Appl. No.: 17/913,189

(22) PCT Filed: Mar. 26, 2021
(86) PCT No.: PCT/US2021/024431

§ 371 Date: Sep. 21, 2022

Related U.S. Application Data
(60) Provisional application No. 63/000,694, filed on Mar. 27, 2020.

(51) Int. Cl. C07K 14/725; C12Q 1/6886; G01N 33/574

(52) U.S. Cl. CPC C07K 14/7051; C12Q 1/6886; G01N 33/574; C12Q 2600/158; A61K 2039/5158

(57) ABSTRACT

Surface proteins predominantly associated with multiple myeloma are identified as potential targets for developing anti-multiple myeloma therapeutics. In accordance with one embodiment antibodies are generated that specifically bind to epitopes of the identified protein that are associated with multiple myeloma cells. These antibodies can then be used to target the delivery of cytotoxic agents to multiple myeloma cells in a patient or used to prepare CAR T-cells for the treatment of multiple myeloma patients.

### CROSS REFERENCE TO RELATED APPLICATIONS

This application claims priority to U.S. Provisional Patent Application No. 63/000,694 filed on Mar. 27, 2020, the disclosure of which is expressly incorporated herein.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 1,490 kilobytes ASCII (text) file named “335022_ST25,” created on Mar. 22, 2021.

### BACKGROUND OF THE DISCLOSURE

Multiple myeloma (MM), also known as plasma cell myeloma, is a cancer of plasma cells, a type of white blood cell that normally produces antibodies. Globally, multiple myeloma affected 488,000 people and resulted in 101,100 deaths in 2015. In the United States, it develops in 6.5 per 100,000 people per year and 0.7% of people are affected at some point in their lives. Without treatment, typical survival is seven months. With current treatments, survival is usually 4-5 years.

Targeting tumor antigens with immunotherapy is rapidly emerging as a promising approach for cancer treatment. This is based on successes of antibody-mediated checkpoint blockade and engineered T cells. In MM, monoclonal antibodies, antibody-drug conjugates, bi-specific antibody constructs, and Chimeric Antigen Receptor (CAR) T-cell therapy targeting BCMA (B-cell maturation antigen) are significantly improving survival in patients with MM. Data from >20 clinical trials involving anti-BCMA CAR T cells have demonstrated that patients with relapsed and/or refractory MM can achieve objective responses.

While tremendous progress in the treatment of Multiple Myeloma (MM) has been made over the past 25 years, myeloma remains an incurable disease, with a particularly poor prognosis for patients with refractory relapsed MM (RRMM) or high-risk cytogenetics. The remarkable success of CD19-Chimeric Antigen Receptor (CAR) T cells in patients with lymphoid malignancies has prompted the development of CAR T cells for treating MM. BCMA (aka TNFRSF17) is the first surface target utilized to generate CAR T cells for patients with RRMM who have undergone at least three prior treatments, including treatment with a proteasome inhibitor and an immunomodulatory agent. Targeting BCMA with antibody conjugates and bispecific T-cell engagers (BiTE; a unique artificial bispecific monoclonal antibody that has two linked, single-chain variable fragments and having a 1+1 antigen-binding valency) has been demonstrated to have efficacy in treating MM. Both BCMA targeting immune-therapies were granted breakthrough status for patients with RRMM by FDA. All these trials demonstrate impressive results with the ability of anti-BCMA CAR T cells to induce deep responses in highly pretreated RRMM, however, despite this, remissions are not sustained and the majority of patients eventually relapse. One of the mechanisms of resistance lies in the antigen loss or downregulation with the emergence of low BCMA or BCMA-negative subclones. Identifying alternative targets is crucial to provide therapeutic options to patients who failed BCMA CAR therapy and/or design combinatorial strategies limiting the risk of antigen escape. One of the most important determinants of the success of CAR T-cell therapy is the choice of the target antigen; an ideal target should be highly expressed on all tumor cells, on cancer stem cells, in most patients, absent in normal counterparts and most organs of the whole body. Given that, studying the myeloma surface proteome is critical to identify additional immunotherapeutic targets and understand the role of an altered surfaceome in the disease biology. In fact, surface proteins may mediate regulatory mechanisms underpinning myeloma manipulation of the bone marrow microenvironment.

To this purpose, one aspect of the present disclosure is directed to the use of high-quality Mass-Spectrometry methodologies and generated integrative bioinformatics tools to unbiasedly and accurately map the cell surface of MM cell lines and primary MM patient samples bearing distinct genetic backgrounds. These helped overcome the challenge of studying surface proteins that present with low abundance, high hydrophobicity and heavy post-translational modifications compared to intracellular proteins. In accordance with the present disclosure methods are provided for identifying additional targets that can be used to design alternative CAR T-cell therapeutics beyond BCMA.

### SUMMARY

The present disclosure is directed to the identification of candidate cell surface antigens that can be utilized as immuno-therapeutic targets for developing novel drugs including antibodies and CAR T cells for diagnosing and treating patients with Multiple Myeloma. The antibodies of the present invention can also be used to identify disease markers for diagnostic purposes like flow cytometry. In accordance with one embodiment the identified targets disclosed herein have the potential to serve as marker for identifying and treating those Multiple Myeloma (MM) patients that display surface targets significantly associated with features of poor prognosis (i.e. associated with high-risk MM).

In accordance with one embodiment a method is provided for identifying target cell surface antigens that are specific for multiple myeloma cells and can be used as immuno-therapeutic targets. In one embodiment the method comprises performing surface-specific proteomic analyses of multiple myeloma cell lines bearing distinct genetic abnormalities using biotin labeling followed by Mass-Spectrometry analysis (MS). In a further embodiment RNA-seq datasets of MM patients are investigated to identify surface proteins whose gene expression is elevated in MM patients relative to normal tissues. In one embodiment, a method is provided for identifying target cell surface antigens that are specific for multiple myeloma cells wherein the method comprises Mass-Spectrometry analysis of surface labeled proteins and analysis of RNA-seq datasets of MM patients to identify proteins common to both analysis as target cell surface antigens.

In accordance with one embodiment cell surface polypeptides having the sequence of SEQ ID NO: 1 through SEQ ID NO: 155 have been identified as being associated with MM cells. Accordingly, the peptides of SEQ ID NO: 1-155 represent targets for immuno-based therapeutic strategies for treating MM. In accordance with one embodiment an antibody that specifically binds to a polypeptide selected from the group consisting of SEQ ID NO: 1-155 is provided. In accordance with one embodiment the antibody is a monoclonal antibody. Also encompassed by the present invention are antibody fragments wherein the fragment retains the ability to bind to the same epitope as the whole antibody.

In accordance with one embodiment cell surface polypeptides IL12RB1, SLC5A3, CCR1, ANKH, IL27RA, S1PR4, TLR1, KCNA3, PSEN2, BCMA, IL2RG, LILRB4, IL6R, ITGB7, LRP10, FCRL3, IFNGR1, SLAMF6, SLCO3A1, LILRB1, PLXNA3, SLC17A5, CD28, LAX1, NEMP1, TMEM154, SEMA4A, C10orf54, ITM2C, LY9, SLAMF7, ITGA4, LRRC8A, LRRC8D, CD320, KCNN4, PLXNC1, CD37, SELPLG, DAGLB, ABCC4, ADAMS, CD4, CD180, CD48, CD40, MCUR1, ABCC5, IL6ST, LRP8, SLC5A6, SLC7A6, HLA-F, ICAM2, LEPROT, ITGAL, TMEM63A, CMTM7, IL10RB, NDC1, PTPRCAP, ANTXR2, ABCA7, FCGR2B, ACVR1B, STS, ABHD12, TNFRSF10A, HVCN1, SLC39A10, EMP3, ABCC1, SLC26A6, SLC6A6, CCR10, SLC30A1, SLC231A1, ADCY3, IFNAR1, PTPRJ, CLDND1, SLC30A5, SLC6A9, ADAM15, IGF2R, INSR, NOTCH2, CD53, SLC12A9, SLC15A4, CEMIP2, ADAM17, MPZL1 and TACI have been identified as being associated with MM cells. Accordingly, these peptides represent targets for immuno-based therapeutic strategies for treating MM. In accordance with one embodiment an antibody that specifically binds to one of these polypeptides is provided. In accordance with one embodiment the antibody is a monoclonal antibody. Also encompassed by the present invention are antibody fragments wherein the fragment retains the ability to bind to the same epitope as the whole antibody.

In accordance with one embodiment cell surface polypeptides IL12RB1, CCR1, LILRB4, FCRL3, IFNGR1, SLAMF6, LAX1, SEMA4A, ITGA4, LRRC8D and CD320 have been identified as being associated with MM cells. Accordingly, these peptides represent targets for immuno-based therapeutic strategies for treating MM. In accordance with one embodiment an antibody that specifically binds to one of these polypeptides is provided. In accordance with one embodiment the antibody is a monoclonal antibody. Also encompassed by the present invention are antibody fragments wherein the fragment retains the ability to bind to the same epitope as the whole antibody.

In accordance with one embodiment an antibody or antibody fragment is provided that binds to a polypeptide selected from the group consisting of SEQ ID NO: 1-115. In accordance with one embodiment the antibody or antibody fragment binds to a polypeptide selected from the group consisting of SEQ ID NO: 1-10. In accordance with one embodiment the antibody or antibody fragment binds to a polypeptide selected from the group consisting of SEQ ID NO: 11-20. In accordance with one embodiment the antibody or antibody fragment binds to a polypeptide selected from the group consisting of SEQ ID NO: 21-30. In accordance with one embodiment the antibody or antibody fragment binds to a polypeptide selected from the group consisting of SEQ ID NO: 31-40. In accordance with one embodiment the antibody or antibody fragment binds to a polypeptide selected from the group consisting of SEQ ID NO: 41-50. In accordance with one embodiment the antibody or antibody fragment binds to a polypeptide selected from the group consisting of SEQ ID NO: 51-60. In accordance with one embodiment the antibody or antibody fragment binds to a polypeptide selected from the group consisting of SEQ ID NO: 61-70. In accordance with one embodiment the antibody or antibody fragment binds to a polypeptide selected from the group consisting of SEQ ID NO: 71-80. In accordance with one embodiment the antibody or antibody fragment binds to a polypeptide selected from the group consisting of SEQ ID NO: 81-90. In accordance with one embodiment the antibody or antibody fragment binds to a polypeptide selected from the group consisting of SEQ ID NO: 91-100. In accordance with one embodiment the antibody or antibody fragment binds to a polypeptide selected from the group consisting of SEQ ID NO: 101-110. In accordance with one embodiment the antibody or antibody fragment binds to a polypeptide selected from the group consisting of SEQ ID NO: 111-120. In accordance with one embodiment the antibody or antibody fragment binds to a polypeptide selected from the group consisting of SEQ ID NO: 121-130. In accordance with one embodiment the antibody or antibody fragment binds to a polypeptide selected from the group consisting of SEQ ID NO: 131-140. In accordance with one embodiment the antibody or antibody fragment binds to a polypeptide selected from the group consisting of SEQ ID NO: 141-147.

In accordance with one embodiment a monoclonal antibody is provided that specifically binds to a polypeptide selected from the group consisting of SEQ ID NO: 168-208. In accordance with one embodiment a monoclonal antibody is provided wherein the antibody specifically binds to a polypeptide having as least 90, 95 or 99% sequence identity to a polypeptide selected from the group consisting of SEQ ID NO: 19, SEQ ID NO: 20, SEQ ID NO: 37, SEQ ID NO: 42, SEQ ID NO: 54, SEQ ID NO: 56, SEQ ID NO: 57, SEQ ID NO: 79, SEQ ID NO: 112 and SEQ ID NO: 104.

In accordance with one embodiment a monoclonal antibody is provided wherein the antibody specifically binds to a polypeptide having as least 90, 95 or 99% sequence identity to a polypeptide selected from the group consisting of CCR1 (SEQ ID NO: 60), CD320 (SEQ ID NO: 56), FCRL3(SEQ ID NO: 57), IFNGR1 (SEQ ID NO: 79), IL12RB1 (SEQ ID NO: 19), ITGA4 (SEQ ID NO: 42), LILRB4 (SEQ ID NO: 20), LRRC8D (SEQ ID NO: 112), SEMA4A (SEQ ID NO: 104), SLAMF6 (SEQ ID NO: 37) and TNFRSF17 (SEQ ID NO: 167).

In accordance with one embodiment a monoclonal antibody is provided wherein the antibody specifically binds to a polypeptide having as least 90, 95 or 99% sequence identity to a polypeptide selected from the group consisting of IL12RB1, CCR1(SEQ ID NO: 60), LILRB4 (SEQ ID NO: 20), FCRL3 (SEQ ID NO: 57), IFNGR1 (SEQ ID NO: 79), SLAMF6 (SEQ ID NO: 37), SEMA4A (SEQ ID NO: 104), ITGA4 (SEQ ID NO: 42), LRRC8D (SEQ ID NO: 112) and CD320 (SEQ ID NO: 56).

In accordance with one embodiment a monoclonal antibody is provided wherein the antibody specifically binds to a polypeptide having as least 90, 95 or 99% sequence identity to a polypeptide selected from the group consisting of SEQ ID NO: 1-155 or 168-208, optionally wherein the polypeptide selected from the group consisting of SEQ ID NO: 168-208.

In accordance with one embodiment any of the antibodies disclosed herein optionally further comprises a detectable marker and/or a cytotoxic agent linked to the antibody.

In accordance with one embodiment a composition is provided comprising 2, 3, 4, 5, 6, 7, 8, 9 or more of any of the antibodies disclosed herein wherein the plurality of antibodies each binds a different epitope displayed by the polypeptides of SEQ ID NO: 1 through SEQ ID NO: 155.

In accordance with one embodiment any of the antibodies disclosed herein is linked to a solid support. In one embodiment any of the antibodies disclosed herein is covalently linked to a detectable label. In one embodiment any of the antibodies disclosed herein is covalently linked a cytotoxic agent.

In accordance with one embodiment a chimeric antigen receptor (CAR) is provided comprising

an antibody, or antigen binding fragment thereof, that binds one or more epitopes of a polypeptide selected from the group consisting of SEQ ID NO: 1-155,

a transmembrane domain; and

aT-cell antigen receptor chain, wherein the transmembrane domain links the an antibody, or antigen binding fragment thereof to the T-cell antigen receptor chain.

In accordance with one embodiment a chimeric antigen receptor (CAR) is provided comprising

an antibody single-chain variable fragment that specifically binds to one or more epitopes of a polypeptide selected from the group consisting of SEQ ID NO: 1-155, a transmembrane domain; and a T-cell antigen receptor chain, wherein the transmembrane domain links the antibody single-chain variable fragment to the T-cell antigen receptor chain. In one embodiment the T-cell antigen receptor chain of the chimeric antigen receptor comprises a CD3ζ chain (zeta-chain). In one embodiment the chimeric antigen receptor specifically binds to a polypeptide selected from the group consisting of SEQ ID NO: 1-25, or a polypeptide selected from the group consisting of SEQ ID NO: 25-50, or polypeptide selected from the group consisting of SEQ ID NO: 50-75, or a polypeptide selected from the group consisting of SEQ ID NO: 75-100, or a polypeptide selected from the group consisting of SEQ ID NO: 100-125. In accordance with one embodiment the chimeric antigen receptor further comprises a hinge region located between the antibody single-chain variable fragment and the transmembrane domain.

In accordance with one embodiment a modified T-cell (CAR T-cell) is provided wherein the T-cell has been transformed to express a chimeric antigen receptor of the present disclosure that specifically binds to a polypeptide selected from the group consisting of SEQ ID NO 1-155. In one embodiment the T-cell antigen receptor chain of the chimeric antigen receptor is a CD3ζ chain (zeta-chain).

In one embodiment a pharmaceutical composition is provided comprising any of the antibodies, chimeric antigen receptors or CAR T-cells as disclosed herein, optionally including a pharmaceutically acceptable carrier. The pharmaceutical composition can be formulated using standard techniques for any suitable route of administration including intravenously or intraperitoneally.

In accordance with one embodiment a method for treating multiple myeloma is provided. In one embodiment the method comprises administering a therapeutic amount of any of the antibodies, chimeric antigen receptors or CAR T-cells of the present disclosure to a patient in need of such treatment. In one embodiment the compositions is administered as a pharmaceutical composition comprising any of the antibodies, chimeric antigen receptors or CAR T-cells as disclosed herein and a pharmaceutically acceptable carrier.

In accordance with one embodiment a method of treating MM comprises administering CAR T-cells that express chimeric antigen receptors that specifically bind to a polypeptide selected from the group consisting of SEQ ID NO: 1-155. In one embodiment the treatment is conducted in conjunction with other known therapeutic treatments known to the skilled practitioner, including the co-administration of CAR T-cells directed to BCMA or other known surface candidate targets in MM including the antigens SLAMF7, CD38, GPRC5D, ITGB7, CD229, CD56, TACI, CD19 and CD70.

### BRIEF DESCRIPTION OF THE DRAWINGS

FIGS. 1A-1D represent the steps for identifying target MM genes, FIG. 1A is a schematic representation of the biotinylation of the surface proteins of 7 different MM cell lines followed by Spectrometry analysis. Each cell line bears unique combinations of chromosomal rearrangements and p53 mutations as shown. This analysis led to the identification of 5,454 uniprot IDs corresponding to 4761 proteins FIG. 1B is a schematic representation of the integrated database used to generated for cell surface molecule annotation. For each repository we indicated the methodology used for cell surface molecule annotation and relative size. This also served as a scoring system with 0 denoting a protein not at the cell surface location and 5 a protein detected in all five repositories. The number of IDs per score is also indicated. 16,462 transcripts derived from 904 patients with multiple myeloma were annotated for cell surface molecules. FIG. 1C presents a Venn Diagram showing the overlap between cell surface molecules with a score equal or higher than 3 as detected by Mass-Spectrometer analysis in cell lines and RNA-seq in primary patient samples. As shown in FIG. 1D expression levels of the cell surface molecules was analyzed and by excluding molecules with an expression below 1 SD from the average patient gene expression 326 surface proteins were selected for further analyses.

FIGS. 2A-2C: Enrichment analysis of candidate targets. 326 surface proteins selected in FIG. 1D were analyzed by STRING. A significant number of edges was identified (490) that is higher than expected (109) with an average local functional and/or physical local clustering coefficient of 0.326 and a PPI enrichment p value <1.0e-16. In yellow the largest cluster. The largest cluster (227/326 proteins) involves proteins with a functional enrichment related to immune pathways (FIG. 2A). The other two clusters involve transporters and adhesion molecules FIG. 2B). Additional enrichment analysis of the largest cluster by the KEGG collection is shown in FIG. 2C. Further enrichment analysis of the largest cluster by the Reactome collection is shown.

FIG. 3 provides a Venn Diagram overlapping the targets with a potential biological relevance (227 molecules involved in immune-related pathways) and therapeutic relevance (94 molecules with minimal expression in normal tissues). 67 common targets are common. 24 out of those 67 targets presented the most favorable expression profile in primary patients and were used for validation in patient samples including: CCR1, CD28, CD320, FCRL3, IFNGR1, IL12RB1. IL27RA. IL2RG, IL6R, ITGA4, KCNN4, LAX1, LILRB1, LILRB4, LRRC8A, LRRC8D, PLXNA3, PLXNC1, S1PR4, SELPLG, SEMA4A, SLAMF6, TLR1 and BCMA.

### DETAILED DESCRIPTION

### Definitions

Unless defined otherwise, all technical and scientific terms used herein have the same meanings as commonly understood by one of ordinary skill in the art to which this invention belongs.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein the terms “native” or “natural” define a condition found in nature. A “native DNA sequence” is a DNA sequence present in nature that was produced by natural means but not generated by genetic engineering (e.g., using molecular biology/transformation techniques)

As used herein the term “solid support” relates to a solvent insoluble substrate that is capable of forming linkages (preferably covalent bonds) with soluble molecules. The support can be either biological in nature, such as, without limitation, a cell or bacteriophage particle, or synthetic, such as, without limitation, an acrylamide derivative, glass, plastic, agarose, cellulose, nylon, silica, or magnetized particles. The support can be in particulate form or a monolythic strip or sheet. The surface of such supports may be solid or porous and of any convenient shape.

The term “linked” or like terms refers to the connection between two groups. The linkage may comprise a covalent, ionic, or hydrogen bond or other interaction that binds two compounds or substances to one another.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

“Therapeutic agent,” “pharmaceutical agent” or “drug” refers to any therapeutic or prophylactic agent which may be used in the treatment (including the prevention, diagnosis, alleviation, or cure) of a malady, affliction, disease or injury in a patient.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes alleviating the symptoms associated with a specific disorder or condition and/or preventing or eliminating said symptoms. For example, treating cancer includes preventing or slowing the growth and/or division of cancer cells as well as killing cancer cells.

As used herein, the term “antibody” refers to a polyclonal or monoclonal antibody or a binding fragment thereof such as Fab, F(ab′)₂ and Fv fragments. Antibodies as disclosed herein include, but are not limited to, monoclonal, multispecific, human or chimeric antibodies, single chain antibodies, Fab fragments, F(ab′) fragments, anti-idiotypic (anti-Id)antibodies (including, e.g., anti-Id antibodies to antibodies of the invention), intracellularly-made antibodies (i.e., intrabodies), and epitope-binding fragments of any of the above. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule

As used herein, the term “biologically active fragments” of the antibodies described herein encompasses natural or synthetic portions of the respective full-length antibody that retain the capability of specific binding to the target epitope.

As used herein, the term “parenteral” includes administration subcutaneously, intravenously or intramuscularly.

As used herein the characterization of expression level such as “high expression” is based on the parameters established in Perna et al., Cancer Cell 32, 506-519. Oct. 9, 2017, the teachings of which are expressely incorporated herein. By merging 3 proteomic databases a cut-off for low medium and high expression was established and was used in the context of the present invention.

### EMBODIMENTS

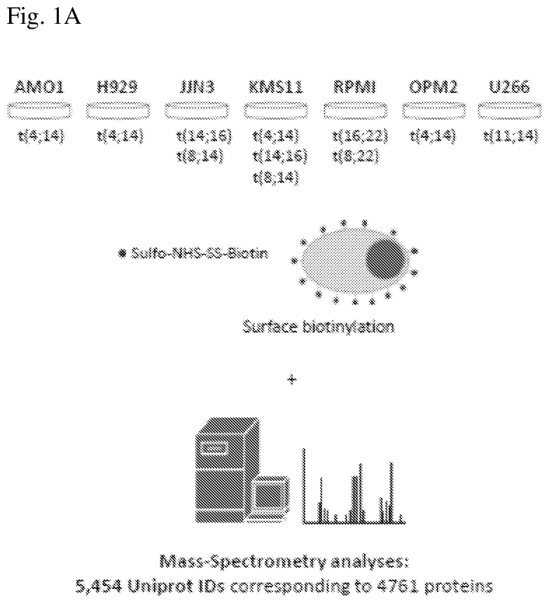

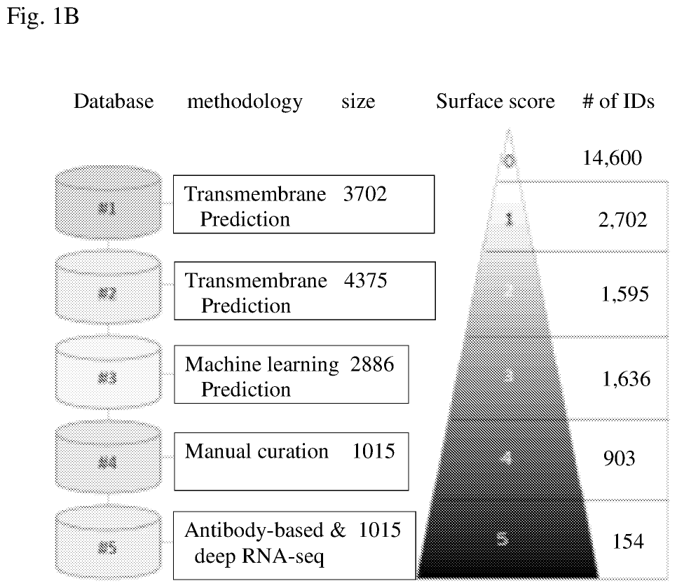

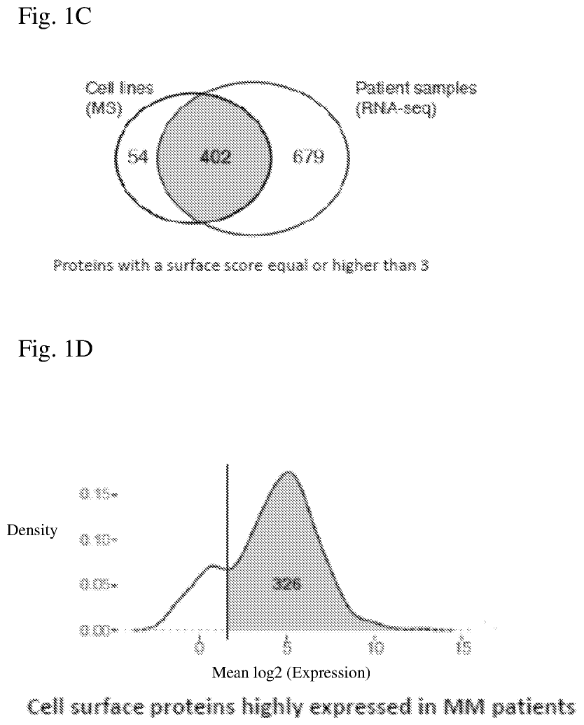

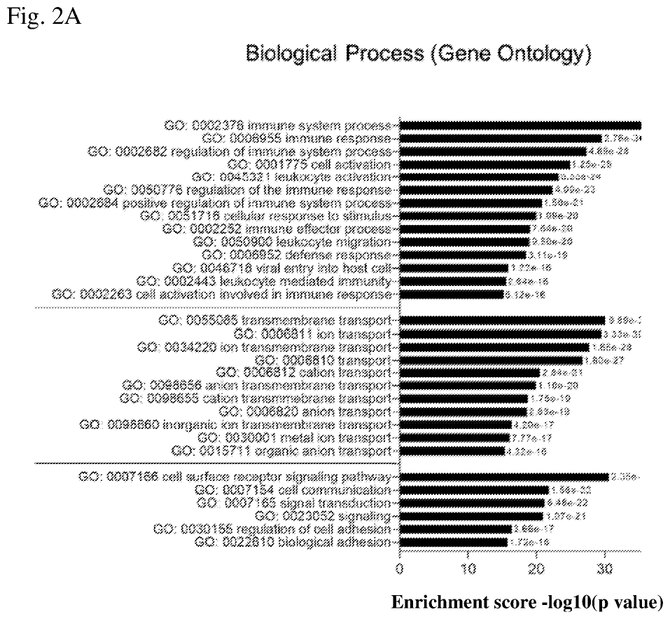

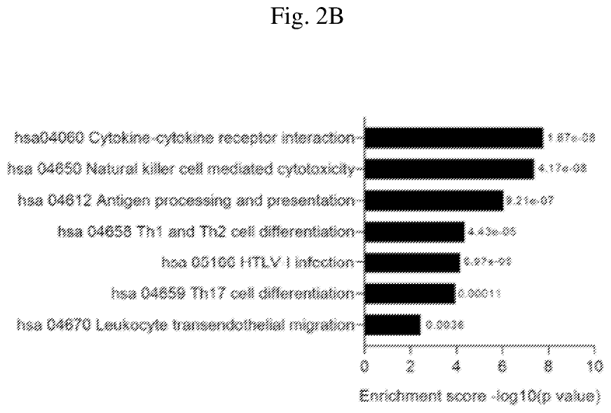

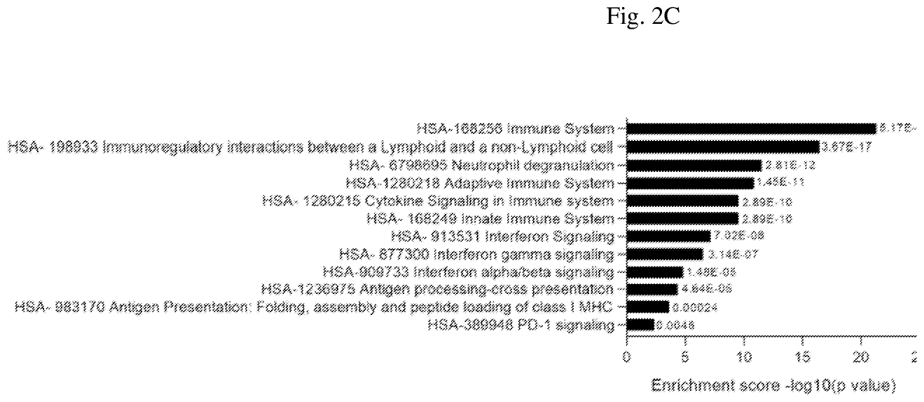

The present disclosure is based on applicant's analysis of the expression of cell surface candidate targets in MM. Biotinylation of the surface proteins of 7 different MM cell lines followed by Spectrometry analysis led to the identification of 5,454 uniprot IDs corresponding to 4761 proteins. As detailed in FIG. 1B an integrated database was used to generate cell surface molecule annotation. A combination of cell surface molecule annotation and exclusion based on expression levels (see FIGS. 1C and 1D) identified 326 surface proteins for further analysis by STRING.

A heatmap reveals the protein annotation of 94 selected targets in several normal tissues and organs of the whole body. These molecules were selected by merging the Human Protein Atlas, Human Protein Map and Proteomics database as previously reported (Perna F et al., Cancer Cell 2017). Molecules with high expression in any normal tissue except hematopoietic tissues were excluded. Molecules with available annotation in less than 2 out of the 3 proteomic databases were excluded. The 94 targets include the cell surface polypeptides IL12RB1, SLC5A3, CCR1, ANKH, IL27RA, S1PR4, TLR1, KCNA3, PSEN2, BCMA, IL2RG, LILRB4, IL6R, ITGB7, LRP10, FCRL3, IFNGR1, SLAMF6, SLCO3A1, LILRB1, PLXNA3, SLC17A5, CD28, LAX1, NEMP1, TMEM154, SEMA4A, C10orf54, ITM2C, LY9, SLAMF7, ITGA4, LRRC8A, LRRC8D, CD320, KCNN4, PLXNC1, CD37, SELPLG, DAGLB, ABCC4, ADAMS, CD4, CD180, CD48, CD40, MCUR1, ABCC5, IL6ST, LRP8, SLC5A6, SLC7A6, HLA-F, ICAM2, LEPROT, ITGAL, TMEM63A, CMTM7, IL10RB, NDC1, PTPRCAP, ANTXR2, ABCA7, FCGR2B, ACVR1B, STS, ABHD12, TNFRSF10A, HVCN1, SLC39A10, EMP3, ABCC1, SLC26A6, SLC6A6, CCR10, SLC30A1, SLC231A1, ADCY3, IFNAR1, PTPRJ, CLDND1, SLC30A5, SLC6A9, ADAM15, IGF2R, INSR, NOTCH2, CD53, SLC12A9, SLC15A4, CEMIP2, ADAM17, MPZL1 and TACI.

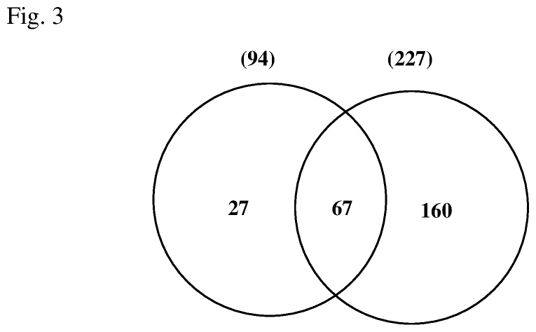

A Venn Diagram overlapping the targets with a potential biological relevance (227 molecules involved in immune-related pathways) and therapeutic relevance (94 molecules with minimal expression in normal tissues) revealed 67 common targets (see FIG. 3). 24 out of those 67 targets presented the most favorable expression profile in primary patients and were used for validation in patient samples including: CCR1, CD28, CD320, FCRL3, IFNGR1, IL12RB1. IL27RA. IL2RG, IL6R, ITGA4, KCNN4, LAX1, LILRB1, LILRB4, LRRC8A, LRRC8D, PLXNA3, PLXNC1, S1PR4, SELPLG, SEMA4A, SLAMF6, TLR1 and BCMA.

Further analysis as described in Example 2 has further identified the following 12 genes that encode products that in some aspects are targets for the generation of immunotherapeutics: CCR1 (SEQ ID NO: 156), CD320 (SEQ ID NO: 157), FCRL3(SEQ ID NO: 158), IFNGR1 (SEQ ID NO: 159), IL12RB1 (SEQ ID NO: 160), ITGA4 (SEQ ID NO: 161), LAX1 (SEQ ID NO: 162), LILRB4 (SEQ ID NO: 163), LRRC8D (SEQ ID NO: 164), SEMA4A (SEQ ID NO: 165), SLAMF6 (SEQ ID NO: 166), and TNFRSF17 (SEQ ID NO: 167).

Western Blot analysis identified the expression of 11 selected targets in in 25 MM patients relative to normal cord blood CD34+ cells revealing these proteins to be of particular interest as targets. BCMA was used as a control. VCP was used as loading control. Specifically, the identified proteins include IL12RB1, CCR1, LILRB4, FCRL3, IFNGR1, SLAMF6, LAX1, SEMA4A, ITGA4, LRRC8D and CD320 by western blot analysis.

In accordance with at least one embodiment Mass-Spectrometer analysis reveals 5,454 Uniprot ID and MMRF Compass revealed 16,462 transcripts. Common targets of this analysis revealed a total of 401 proteins with surface score greater than or equal to (=>) 3. Overlap with high expressors (average expression is greater than (>) median) in primary patient samples (MMRF) was determined and the average expression of each gene was calculated; low expressing genes (1 SD below the mean) were removed and genes having expression over the cutoff were selected identifying 326 surface proteins.

Annotation in normal tissues from at least 2 out of 3 databases (HPA, HPM and PDB) and exclusion of proteins with high expression in any tissue except hematopoietic tissues identified 94 out of the 326 targets. Analysis of immune-related+therapeutic value reduced this number to 67 targets. Analysis in high-risk cytogenetics excluded targets with decreased expression in standard risk vs high-risk and validation in primary MM samples produce 11 targets identified as CCR1 (SEQ ID NO: 156), CD320 (SEQ ID NO: 157), FCRL3 (SEQ ID NO: 158), IFNGR1 (SEQ ID NO: 159), IL12RB1 (SEQ ID NO: 160), ITGA4 (SEQ ID NO: 161), LAX1 (SEQ ID NO: 162), LILRB4 (SEQ ID NO: 163), LRRC8D (SEQ ID NO: 164), SEMA4A (SEQ ID NO: 165), and SLAMF6 (SEQ ID NO: 166).

In accordance with at least one embodiment an antibody is provided that specifically binds to a polypeptide comprising a sequence selected from the group consisting of SEQ ID NO: 1-155. In some embodiments an antibody is provided that specifically binds to a polypeptide comprising a sequence selected from the group consisting of (SEQ ID NO: 156), (SEQ ID NO: 157), (SEQ ID NO: 158), (SEQ ID NO: 159), (SEQ ID NO: 160), (SEQ ID NO: 161), (SEQ ID NO: 162), (SEQ ID NO: 163), (SEQ ID NO: 164), (SEQ ID NO: 165), and (SEQ ID NO: 166). Such antibodies can be linked to detectable markers for diagnostic purposes. Alternatively, the antibodies can be linked to cytotoxic agents and the conjugated antibodies can be administered to patients for targeted delivery of cytotoxins to MM cells.

In at least one embodiment antibodies generated against the peptides disclosed in Table 1 (SEQ ID NO: 1-155), or a polypeptide comprising a sequence selected from the group consisting of (SEQ ID NO: 156), (SEQ ID NO: 157), (SEQ ID NO: 158), (SEQ ID NO: 159), (SEQ ID NO: 160), (SEQ ID NO: 161), (SEQ ID NO: 162), (SEQ ID NO: 163), (SEQ ID NO: 164), (SEQ ID NO: 165), and (SEQ ID NO: 166) can be used to generate an antibody single-chain variable fragment which can then be used to prepare a chimeric antigen receptor (CAR). The antibody single-chain variable fragment is a chimeric protein made up of the light (VL) and heavy (VH) chains of immunoglobins, connected with a short linker peptide. In accordance with the present disclosure the VL and VH regions are selected in advance for their binding ability to a target antigen selected from the polypeptides of SEQ ID NO: 1-155, or a polypeptide comprising a sequence selected from the group consisting of (SEQ ID NO: 156), (SEQ ID NO: 157), (SEQ ID NO: 158), (SEQ ID NO: 159), (SEQ ID NO: 160), (SEQ ID NO: 161), (SEQ ID NO: 162), (SEQ ID NO: 163), (SEQ ID NO: 164), (SEQ ID NO: 165), and (SEQ ID NO: 166). In some embodiments the linker between the VL and VH regions consists of hydrophilic residues with stretches of glycine and serine in it for flexibility as well as stretches of glutamate and lysine for added solubility.

In some aspects, the antibody single-chain variable fragment can then be covalently linked to an intracellular immune cell signaling domain typically through a transmembrane domain to create a chimeric antigen receptor (CAR). In some aspects, the immune cell signaling domain can be a T-cell, NK cell, macrophage, and/or a myeloid cell. For example, in some aspects, the antibody single-chain variable fragment can then be covalently linked to an intracellular T-cell signaling domain typically through a transmembrane domain to create a chimeric antigen receptor (CAR). Such CARs when expressed in immune cells such as T-cells, NK cells, macrophages, and/or a myeloid cells can provide the immune cells a new ability to target a specific protein. Accordingly, CAR T-cells, NK cells, macrophages, and/or myeloid cells comprising CARs that specifically bind to polypeptides selected from the group consisting of SEQ ID NO: 1-155, or a polypeptide comprising a sequence selected from the group consisting of (SEQ ID NO: 156), (SEQ ID NO: 157), (SEQ ID NO: 158), (SEQ ID NO: 159), (SEQ ID NO: 160), (SEQ ID NO: 161), (SEQ ID NO: 162), (SEQ ID NO: 163), (SEQ ID NO: 164), (SEQ ID NO: 165), and (SEQ ID NO: 166) can be used to treat MM patients. In particular, in some embodiments CAR T-cells are prepared by isolating the patient's own cells and transforming T-cells with nucleic acid sequences encoding CAR having specificity to a polypeptide selected from the group consisting of SEQ ID NO: 1-155. In particular, in some embodiments the CAR T-cells are prepared by providing allogeneic T-cells and transforming T-cells with nucleic acid sequences encoding CAR having specificity to a polypeptide selected from the group consisting of SEQ ID NO: 1-155, 156-167, and 168-208, and optionally selected from the group consisting of (SEQ ID NO: 156), (SEQ ID NO: 157), (SEQ ID NO: 158), (SEQ ID NO: 159), (SEQ ID NO: 160), (SEQ ID NO: 161), (SEQ ID NO: 162), (SEQ ID NO: 163), (SEQ ID NO: 164), (SEQ ID NO: 165), and (SEQ ID NO: 166). It is understood that for any of the above cell types, either autologous or allogeneic approaches may be utilized. In accordance with embodiments, the method of treating a patient with MM comprises administering 1, 2, 3, 4, 5 or more CAR T-cells, CAR NK cells, CAR macrophages, and/or CAR myeloid cells to a MM patient in need to therapy, wherein each of the CAR T-cells targets a different antigen present on a polypeptide selected from the group consisting of SEQ ID NO: 1-155, or a polypeptide comprising a sequence selected from the group consisting of (SEQ ID NO: 156), (SEQ ID NO: 157), (SEQ ID NO: 158), (SEQ ID NO: 159), (SEQ ID NO: 160), (SEQ ID NO: 161), (SEQ ID NO: 162), (SEQ ID NO: 163), (SEQ ID NO: 164), (SEQ ID NO: 165), and (SEQ ID NO: 166).

In accordance with at least one embodiment an antibody is provided that specifically binds to a gene product of CCR1 (SEQ ID NO: 156), CD320 (SEQ ID NO: 157), FCRL3(SEQ ID NO: 158), IFNGR1 (SEQ ID NO: 159), IL12RB1 (SEQ ID NO: 160), ITGA4 (SEQ ID NO: 161), LAX1 (SEQ ID NO: 162), LILRB4 (SEQ ID NO: 163), LRRC8D (SEQ ID NO: 164), SEMA4A (SEQ ID NO: 165), SLAMF6 (SEQ ID NO: 166), and TNFRSF17 (SEQ ID NO: 167). In one embodiment an antibody is provided that specifically binds to a polypeptide comprising a sequence selected from the group consisting of SEQ ID NO: 168-208. Such antibodies can be linked to detectable markers for diagnostic purposes. Alternatively, the antibodies can be linked to cytotoxic agents and the conjugated antibodies can be administered to patients for targeted delivery of cytotoxins to MM cells.

In some embodiments antibodies generated against the peptides disclosed in Table 1 (SEQ ID NO: 168-208) can be used to generate an antibody single-chain variable fragment which can then be used to prepare a chimeric antigen receptor (CAR). The antibody single-chain variable fragment is a chimeric protein made up of the light (VL) and heavy (VH) chains of immunoglobins, connected with a short linker peptide. In accordance with the present disclosure the VL and VH regions are selected in advance for their binding ability to a target antigen selected from the polypeptides of SEQ ID NO: 168-208. In one embodiment the linker between the VL and VH regions consists of hydrophilic residues with stretches of glycine and serine in it for flexibility as well as stretches of glutamate and lysine for added solubility.

The antibody single-chain variable fragment can then be covalently linked to an intracellular T-cell signaling domain typically through a transmembrane domain to create a chimeric antigen receptor (CAR). Such CARs when expressed in T-cells can provide T cells a new ability to target a specific protein. Accordingly, CAR T-cells comprising CARs that specifically bind to polypeptides selected from the group consisting of SEQ ID NO: 168-208 can be used to treat MM patients. In particular, in some embodiments the CAR T-cells are prepared by isolating the patient's own cells and transforming T-cells with nucleic acid sequences encoding CAR having specificity to a polypeptide selected from the group consisting of SEQ ID NO: 168-208. In accordance with one embodiment the method of treating a patient with MM comprises administering 1, 2, 3, 4, 5 or more CAR T-cells to a MM patient in need to therapy, wherein each of the CAR T-cells targets a different antigen present on a polypeptide selected from the group consisting of SEQ ID NO: 168-208.

In accordance with one embodiment the transmembrane domain of the CARs of the present disclosure comprises a hydrophobic alpha helix that spans the cell membrane. It anchors the CAR to the plasma membrane, bridging the extracellular antigen recognition domains (i.e., antibody single-chain variable fragment) with the intracellular signaling region. In one embodiment the CAR further comprises a hinge region located between the antigen recognition domains and the transmembrane domain. The ideal hinge enhances the flexibility of the scFv receptor head, reducing the spatial constraints between the CAR and its target antigen. This promotes antigen binding and synapse formation between the CAR-T cells and target cells. In one embodiment the hinge sequences is based on membrane-proximal regions from other immune molecules including IgG, CD8, and CD28.

The intracellular T-cell signaling domain of the CAR when expressed a cell will remain inside the cell. After an antigen is bound to the external antigen recognition domain, CAR receptors cluster together and transmit an activation signal. Then the internal cytoplasmic end of the receptor perpetuates signaling inside the T cell. Normal T cell activation relies on the phosphorylation of immunoreceptor tyrosine-based activation motifs (ITAMs) present in the cytoplasmic domain of CD3-zeta. To mimic this process, in one embodiment the CARS of the present disclosure comprise the CD3-zeta's cytoplasmic domain as the main CAR endodomain component.

T cells also require co-stimulatory molecules in addition to CD3 signaling in order to persist after activation. In a further embodiment, the endodomains of CAR receptors also include one or more chimeric domains from co-stimulatory proteins known to those skilled in the art. Signaling domains from a wide variety of co-stimulatory molecules have been successfully tested, including CD28, CD27, CD134 (OX40), and CD137. In a further embodiment co-stimulatory domains, like CD28 or 4-1BB, CD28-41BB or CD28-OX40, and cytokines, such is IL-2, IL-5, IL-12 can be added to the endodomains of CAR receptors to augment T cell activity.

In accordance with embodiment 1, a method for identifying target multiple myeloma associated surface antigens is provided wherein said method comprises

identifying a plurality of genes that express cell-surface proteins in a first multiple myeloma sample and a second multiple myeloma sample;

selecting nucleic acids from said first multiple myeloma sample that have expression levels higher than a control gene unrelated to hematopoietic cells, and identifying the proteins corresponding to the detected elevated expressed nucleic acids to designate a first pool of selected proteins;

conducting mass spec analysis on proteins isolated from said second myeloma sample to identify proteins that are present in higher concentration in said second multiple myeloma relative to normal tissues, wherein such proteins represent a second pool of selected proteins;

excluding proteins with high expression in brain, spinal cord, gut, liver and kidney from said first and second pools to produce a modified first and second pool of proteins; and

identifying proteins common to said first and second modified pool of proteins as target multiple myeloma associated surface antigens.

In accordance with embodiment 2, the method of embodiment 1 is provided wherein said first multiple myeloma sample and a second multiple myeloma sample are taken from the same tissue source.

In accordance with embodiment 3, the method of embodiment 1 is provided wherein said first multiple myeloma sample is a nucleic acid pool of expressed genes from MM patients and the second multiple myeloma sample represents proteins expressed in MM cell lines.

In accordance with embodiment 4, the method of any one of embodiments 1-3 is provided wherein the target multiple myeloma associated surface antigen has an expression level in a normal tissue sample that is more than about one standard deviation below the normal peak of the protein expression level distribution of the normal tissue sample.

In accordance with embodiment 5, the method of any one of embodiments 1˜4 wherein mRNA is measured to determine the expression level of the nucleic acids used to identify proteins for the first pool of selected proteins.

In accordance with embodiment 6, the method of any one of embodiments 1-5 is provided wherein proteins are identified as cell surface proteins based on the use of an integrative computational tool assigning a score relative to five published databases disclosed in Example 2.

In accordance with embodiment 7, the method of any one of embodiments 1-5 is provided wherein said first pool of proteins is identified by

analyzing an RNA-seq dataset from Multiple Myeloma patients to identify surface targets based on the use of an integrative computational tool assigning a score relative to five published databases disclosed in Example 2;

removing low expressing genes (1 SD below the mean); and

excluding proteins with high expression in any normal tissue except hematopoietic tissues; wherein the remaining proteins constitute said first pool of proteins.

In accordance with embodiment 8, a method for identifying target multiple myeloma associated surface antigens is provided wherein said method comprises

analyzing an RNA-seq dataset from Multiple Myeloma patients to identify surface targets based on the use of an integrative computational tool assigning a score relative to five published databases disclosed in Example 2;

removing low expressing genes (1 SD below the mean); and

excluding proteins with high expression in any normal tissue except hematopoietic tissues; wherein the remaining proteins constitute target multiple myeloma associated surface antigens.

In accordance with embodiment 9, a monoclonal antibody that specifically binds to a polypeptide selected from the group consisting of SEQ ID NO: 1-155 or 168-208 is provided or a monoclonal antibody that specifically binds to a polypeptide having at least 90, 95, or 99% sequence identity with a polypeptide selected from the group consisting of SEQ ID NO: 1-155 or 168-208.

In accordance with embodiment 10, the monoclonal antibody of embodiment 9 is provided wherein the antibody specifically binds to a polypeptide selected from the group consisting of SEQ ID NO: 168-208.

In accordance with embodiment 11, the monoclonal antibody of embodiment 9 is provided wherein the antibody specifically binds to a polypeptide selected from the group consisting of SEQ ID NO: 19, SEQ ID NO: 20, SEQ ID NO: 37, SEQ ID NO: 42, SEQ ID NO: 54, SEQ ID NO: 56, SEQ ID NO: 57, SEQ ID NO: 79, SEQ ID NO: 112 and SEQ ID NO: 104.

In accordance with embodiment 12, the monoclonal antibody of any one of embodiments 9-11 is provided wherein the antibody specifically binds to a polypeptide having at least 90% sequence identity to a sequence selected from the group consisting of CCR1 (SEQ ID NO: 60), CD320 (SEQ ID NO: 56), FCRL3(SEQ ID NO: 57), IFNGR1 (SEQ ID NO: 79), IL12RB1 (SEQ ID NO: 19), ITGA4 (SEQ ID NO: 42), LILRB4 (SEQ ID NO: 20), LRRC8D (SEQ ID NO: 112), SEMA4A (SEQ ID NO: 104), and SLAMF6 (SEQ ID NO: 37).

In accordance with embodiment 13, the monoclonal antibody of any one of embodiments 9-12 is provided wherein the antibody specifically binds to a polypeptide having at least 90% sequence identity to a sequence selected from the group consisting of SEQ ID NO: 1-155 or 168-208.

In accordance with embodiment 14, the monoclonal antibody of any one of embodiments 9-12 is provided wherein the antibody specifically binds to a polypeptide having at least 95% sequence identity to a sequence selected from the group consisting of SEQ ID NO: 1-155 or 168-208.

In accordance with embodiment 15, the monoclonal antibody of any one of embodiments 9-14 is provided wherein said antibody further comprises a detectable label covalently linked to the antibody.

In accordance with embodiment 16, the monoclonal antibody of any one of embodiments 9-14 is provided wherein said antibody further comprises a cytotoxic agent linked to the antibody.

In accordance with embodiment 17, a chimeric antigen receptor (CAR) is provided comprising an antibody of any one of embodiments 9-15, or antigen binding fragment thereof;

a transmembrane domain; and

an immune cell antigen receptor chain, wherein the transmembrane domain links the an antibody, or antigen binding fragment thereof to the immune cell antigen receptor chain.

In accordance with embodiment 18, a chimeric antigen receptor of embodiment 17 is provided wherein the antibody or antigen binding fragment thereof, specifically binds to a polypeptide selected from the group consisting of SEQ ID NO: 168-208.

In accordance with embodiment 19, a chimeric antigen receptor of embodiment 17 is provided wherein the antibody or antigen binding fragment thereof, specifically binds to a polypeptide having at least 95% sequence identity to a sequence selected from the group consisting of CCR1 (SEQ ID NO: 60), CD320 (SEQ ID NO: 56), FCRL3(SEQ ID NO: 57), IFNGR1 (SEQ ID NO: 79), IL12RB1 (SEQ ID NO: 19), ITGA4 (SEQ ID NO: 42), LILRB4 (SEQ ID NO: 20), LRRC8D (SEQ ID NO: 112), SEMA4A (SEQ ID NO: 104) and SLAMF6 (SEQ ID NO: 37).

In accordance with embodiment 20, a chimeric antigen receptor of embodiment 17 is provided wherein the antibody, or antigen binding fragment thereof, binds one or more epitopes of a polypeptide having at least 90% homology to a sequence selected from the group consisting of SEQ ID NO: 1-155 or 168-208.

In accordance with embodiment 21, a chimeric antigen receptor of embodiment 17 is provided wherein the antibody, or antigen binding fragment thereof, binds one or more epitopes of a polypeptide having at least 95% homology to a sequence selected from the group consisting of SEQ ID NO: 1-155 or 168-208.

In accordance with embodiment 22, a chimeric antigen receptor of embodiment 17 is provided wherein the antibody or antigen binding fragment thereof, specifically binds to a polypeptide selected from the group consisting of SEQ ID NO: 19, SEQ ID NO: 20, SEQ ID NO: 37, SEQ ID NO: 42, SEQ ID NO: 54, SEQ ID NO: 56, SEQ ID NO: 57, SEQ ID NO: 79, SEQ ID NO: 112 and SEQ ID NO: 104.

In accordance with embodiment 23, a chimeric antigen receptor of any one of embodiments 17-22 is provided wherein said antibody or antigen binding fragment comprises an antibody single-chain variable fragment.

In accordance with embodiment 24, a chimeric antigen receptor of embodiment 23 is provided further comprising a hinge region located between the antibody single-chain variable fragment and the transmembrane domain.

In accordance with embodiment 25, a T-cell modified to express the chimeric antigen receptor of any one of embodiments 16-23 is provided.

In accordance with embodiment 26, the T-cell of embodiment 25 is provided wherein the T-cell is a tumor infiltrating leukocyte.

In accordance with embodiment 27, an NK cell modified to express the chimeric antigen receptor of any one of embodiments 17-24 is provided.

In accordance with embodiment 28, a myeloid cell modified to express the chimeric antigen receptor of any one of embodiments 17-24 is provided.

In accordance with embodiment 29, a macrophage modified to express the chimeric antigen receptor of any one of embodiments 17-24 is provided.

In accordance with embodiment 30, T-cell of embodiment 25 or 26 is provided wherein the T-cell antigen receptor chain is the CD3ζ chain (zeta-chain).

In accordance with embodiment 31 a pharmaceutical composition comprising the antibody of any one of embodiments 9-16, chimeric antigen receptor of any one of embodiments 17-24, the T-cell of embodiments 25 or 26, the NK cell of embodiment 27, the myeloid cell of embodiment 28 or the macrophage of embodiment 29.

In accordance with embodiment 32 a method for treating multiple myeloma is provided wherein said method comprises administering the pharmaceutical composition of embodiment 31 to a patient in need of such treatment.

In accordance with embodiment 33 an isolated immunoresponsive cell is provided wherein the cell comprises an antigen recognizing receptor that binds to an antigen present on a polypeptide selected from the group consisting of CCR1 (SEQ ID NO: 60), CD320 (SEQ ID NO: 56), FCRL3(SEQ ID NO: 57), IFNGR1 (SEQ ID NO: 79), IL12RB1 (SEQ ID NO: 19), ITGA4 (SEQ ID NO: 42), LILRB4 (SEQ ID NO: 20), LRRC8D (SEQ ID NO: 112), SEMA4A (SEQ ID NO: 104), and SLAMF6 (SEQ ID NO: 37) or a polypeptide having 90, 95 or 99% sequence identity to any of said proteins.

In accordance with embodiment 34 an isolated immunoresponsive cell of embodiment 33 is provided wherein the antigen is present on a polypeptide selected from the group consisting of IL12RB1 (SEQ ID NO: 19), LILRB4 (SEQ ID NO: 20), SLAMF6 (SEQ ID NO: 37), CCR1 (SEQ ID NO: 60), and CD320 (SEQ ID NO: 56).

In accordance with embodiment 35 an isolated immunoresponsive cell of embodiment 33 or 34 is provided wherein said antigen recognizing receptor is a T cell receptor (TCR), or a chimeric antigen receptor (CAR).

In accordance with embodiment 36 an isolated immunoresponsive cell of embodiment 35 is provided wherein said antigen recognizing receptor is a CAR.

In accordance with embodiment 37 an isolated immunoresponsive cell of embodiment 36 is provided wherein the intracellular signaling domain of said CAR is the CD3C-chain, CD97, CD1 la-CD 18, CD2, ICOS, CD27, CD 154, CD8, OX40, 4-IBB, CD28 signaling domain, or combinations thereof.

In accordance with embodiment 38 an isolated immunoresponsive cell of any one of embodiments 33-37 is provided wherein the cell is selected from the group consisting of a T cell, a Natural Killer (NK) cell, a cytotoxic T lymphocyte (CTL), a regulatory T cell, a Natural Killer T (NKT) cell, a human embryonic stem cell, and a pluripotent stem cell from which lymphoid cells may be differentiated.

In accordance with embodiment 39 an isolated immunoresponsive cell is provided comprising:

(a) a first antigen recognizing receptor that binds to a first antigen, and
(b) a second antigen recognizing receptor that binds to a second antigen, wherein each of the first antigen and the second antigen is selected from the group consisting of CCR1 (SEQ ID NO: 60), CD320 (SEQ ID NO: 56), FCRL3(SEQ ID NO: 57), IFNGR1 (SEQ ID NO: 79), IL12RB1 (SEQ ID NO: 19), ITGA4 (SEQ ID NO: 42), LILRB4 (SEQ ID NO: 20), LRRC8D (SEQ ID NO: 112), SEMA4A (SEQ ID NO: 104), and SLAMF6 (SEQ ID NO: 37), and the first antigen and the second antigen are different.

In accordance with embodiment 40 an isolated immunoresponsive cell of embodiment 37 is provided, wherein each of said antigen recognizing receptor is a T cell receptor (TCR) or a chimeric antigen receptor (CAR), optionally wherein the said antigen recognizing receptor is a CAR.

In accordance with embodiment 41 a method of reducing tumor burden in a subject is provided, comprising administering to the subject an effective amount of the immunoresponsive cell of any one of embodiments 33-40.

### Example 1

### Surface Biotinylation and MS Analysis

As multiple myeloma is characterized by significant molecular heterogeneity, 7 known and distinct MM cell lines were selected for additional analysis. The 7 cell lines are: OPM-2 bearing p53 mutation and t(4;14), NCI-H929 with t(4;14) and 8q+, JJN3 with t(14;16) and t(8;14), KMS11 with p53 null and t(4;14), t(14;16) and t(8;14), U266 p53 mut and t(11;14), AMO-1 with p53 WT and t(12;14) and RPMI-8226 with p53 mut, t(16;22), t(8;22) and KRas mut as disclosed in Table 1.

TABLE 1
MM cell lines used for Mass-Spectrum analysis

| | | | BCMA | |
|---|---|---|---|---|
| | | | expression level | TP53 |
| | Cell Lines | Molecular Genetics | (flow cytometry) | status |
| 1 | OPM-2 | t(4; 14) | +++ | Mut |
| 2 | NCI-H929 | t(4; 14) + 8q+ | +++ | WT |
| 3 | JJN3 | t(14; 16) + t(8; 14) | ++ (60%) | +/− |
| 4 | KMS11 | t(4; 14) + t(14; 16) + t(8; | +++ | Null |
| | | 14) | | |
| 5 | U266 | t(11; 14) | +++ | Mut |
| 6 | AMO-1 | t(12; 14) | +++ | WT |
| 7 | RPMI-8226 | t(16; 22) + t(8; 22) | +++ | Mut |

Surface-specific proteomic analyses of these seven multiple myeloma cell lines bearing distinct genetic abnormalities was conducted using biotin labeling followed by Mass-Spectrometry analysis (MS). Cell surface proteins were labeled with biotin on live cells; biotin-tagged proteins were then captured on avidin beads, digested with trypsin and analyzed by Mass-Spectrometry and data independent annotation (DIA).

More particularly, labeling with biotin was conducted using procedures to ensure that only surface proteins were exposed to the biotinylation reagents. Pierce® Cell Surface Protein Biotinylation and isolation Kit (Thermo Fisher, A44390) was used for labeling and isolation of surface proteins. Briefly, the cells were incubated with the membrane-impermeable Sulfo-NHS-SS-biotin reagent for 10 min at RT, after the incubation excess labeling reagent was removed and the cells were washed several times to remove any unbound labeling reagent before lysis. Biotinylated proteins were captured on Neutravidin resin, and any non-specific bound unbiotinylated proteins were removed by repetitive washing of the resins. Prior to MS analysis, the bound biotinylated proteins on the Neutravidin resins were released from the resin by trypsin cleavage overnight. The digested peptides were desalted and purified using StageTip, desalted peptides were analyzed by LC-MS/MS using 170 mins LC gradient and DIA method on Orbitrap Fusion. Samples specific library (SSL) was generated using equal amount of digested proteins from each sample. MS Raw data were searched with SSL using Spectronaut software. Protein expression quantification was done using Label Free Quantification (LFQ). This generated an unbiased pool of 5,454 Uniprot IDs corresponding to 4,761 proteins (FIG. 1A). Table 2 155 proteins identified by Mass spec analysis.

TABLE 2

| | | Protein |
|---|---|---|
| Protein Name | Gene Name | Sequence |
| Anthrax toxin receptor 2 (Capillary | ANTXR2 CMG2 | SEQ ID NO: 1 |
| morphogenesis gene 2 protein) (CMG-2) | | |
| C-type lectin domain family 1 member A (C- | CLEC1A CLEC1 | SEQ ID NO: 2 |
| type lectin-like receptor 1) (CLEC-1) | UNQ569/PRO1131 | |
| T-cell-specific surface glycoprotein CD28 | CD28 | SEQ ID NO: 3 |
| (TP44) (CD antigen CD28) | | |
| Complement receptor type 2 (Cr2) | CR2 C3DR | SEQ ID NO: 4 |
| (Complement C3d receptor) (Epstein-Barr | | |
| virus receptor) (EBV receptor) (CD antigen | | |
| CD21) | | |
| T-cell surface glycoprotein CD8 beta chain | CD8B CD8B1 | SEQ ID NO: 5 |
| (CD antigen CD8b) | | |
| Y + L amino acid transporter 2 (Cationic | SLC7A6 KIAA0245 | SEQ ID NO: 6 |
| amino acid transporter, y+ system) (Solute | | |
| carrier family 7 member 6) (y(+)L-type | | |
| amino acid transporter 2) (Y + LAT2) (y + LAT- | | |
| 2) | | |
| Solute carrier family 52, riboflavin | SLC52A2 GPR172A | SEQ ID NO: 7 |
| transporter, member 2 (Porcine | PAR1 RFT3 | |
| endogenous retrovirus A receptor 1) (PERV- | | |
| A receptor 1) (Protein GPR172A) (Riboflavin | | |
| transporter 3) (hRFT3) | | |
| Tumor necrosis factor receptor superfamily | CD40 TNFRSF5 | SEQ ID NO: 8 |
| member 5 (B-cell surface antigen CD40) | | |
| (Bp50) (CD40L receptor) (CDw40) (CD | | |
| antigen CD40) | | |
| Sodium-coupled neutral amino acid | SLC38A1 ATA1 NAT2 | SEQ ID NO: 9 |
| transporter 1 (Amino acid transporter A1) | SAT1 SNAT1 | |
| (N-system amino acid transporter 2) | | |
| (Solute carrier family 38 member 1) | | |
| (System A amino acid transporter 1) | | |
| (System N amino acid transporter 1) | | |
| T-cell surface glycoprotein CD4 (T-cell | CD4 | SEQ ID NO: 10 |
| surface antigen T4/Leu-3) (CD antigen CD4) | | |
| Leukocyte antigen CD37 (Tetraspanin-26) | CD37 TSPAN26 | SEQ ID NO: 11 |
| (Tspan-26) (CD antigen CD37) | | |
| CD180 antigen (Lymphocyte antigen 64) | CD180 LY64 RP105 | SEQ ID NO: 12 |
| (Radioprotective 105 kDa protein) (CD | | |
| antigen CD180) | | |
| Adhesion G protein-coupled receptor E2 | ADGRE2 EMR2 | SEQ ID NO: 13 |
| (EGF-like module receptor 2) (EGF-like | | |
| module-containing mucin-like hormone | | |
| receptor-like 2) (CD antigen CD312) | | |
| Myeloid cell surface antigen CD33 (Sialic | CD33 SIGLEC3 | SEQ ID NO: 14 |
| acid-binding Ig-like lectin 3) (Siglec-3) | | |
| (gp67) (CD antigen CD33) | | |
| Phospholipid-transporting ATPase ABCA7 | ABCA7 | SEQ ID NO: 15 |
| (EC 7.6.2.1) (ABCA-SSN) (ATP-binding | | |
| cassette sub-family A member 7) | | |
| (Autoantigen SS-N) (Macrophage ABC | | |
| transporter) | | |
| Sodium-dependent multivitamin | SLC5A6 SMVT | SEQ ID NO: 16 |
| transporter (Na(+)-dependent multivitamin | | |
| transporter) (Solute carrier family 5 | | |
| member 6) | | |
| Leukosialin (GPL115) (Galactoglycoprotein) | SPN CD43 | SEQ ID NO: 17 |
| (GALGP) (Leukocyte sialoglycoprotein) | | |
| (Sialophorin) (CD antigen CD43) [Cleaved | | |
| into: CD43 cytoplasmic tail (CD43-ct) | | |
| (CD43ct)] | | |
| Integrin alpha-L (CD11 antigen-like family | ITGAL CD11A | SEQ ID NO: 18 |
| member A) (Leukocyte adhesion | | |
| glycoprotein LFA-1 alpha chain) (LFA-1A) | | |
| (Leukocyte function-associated molecule 1 | | |
| alpha chain) (CD antigen CD11a) | | |
| Interleukin-12 receptor subunit beta-1 (IL- | IL12RB1 IL12R | SEQ ID NO: 19 |
| 12 receptor subunit beta-1) (IL-12R subunit | IL12RB | |
| beta-1) (IL-12R-beta-1) (IL-12RB1) (IL-12 | | |
| receptor beta component) (CD antigen | | |
| CD212) | | |
| Leukocyte immunoglobulin-like receptor | LILRB1 ILT2 LIR1 | SEQ ID NO: 20 |
| subfamily B member 1 (LIR-1) (Leukocyte | MIR7 | |
| immunoglobulin-like receptor 1) (CD85 | | |
| antigen-like family member J) | | |
| (Immunoglobulin-like transcript 2) (ILT-2) | | |
| (Monocyte/macrophage immunoglobulin- | | |
| like receptor 7) (MIR-7) (CD antigen CD85j) | | |
| T-lymphocyte surface antigen Ly-9 (Cell | LY9 CDABP0070 | SEQ ID NO: 21 |
| surface molecule Ly-9) (Lymphocyte | | |
| antigen 9) (SLAM family member 3) | | |
| (SLAMF3) (Signaling lymphocytic activation | | |
| molecule 3) (CD antigen CD229) | | |
| Disintegrin and metalloproteinase domain- | ADAM28 ADAM23 | SEQ ID NO: 22 |
| containing protein 28 (ADAM 28) (EC | MDCL | |
| 3.4.24.-) (Epididymal metalloproteinase- | | |
| like, disintegrin-like, and cysteine-rich | | |
| protein II) (eMDC II) (Metalloproteinase- | | |
| like, disintegrin-like, and cysteine-rich | | |
| protein L) (MDC-L) | | |
| Epithelial membrane protein 3 (EMP-3) | EMP3 YMP | SEQ ID NO: 23 |
| (Hematopoietic neural membrane protein | | |
| 1) (HNMP-1) (Protein YMP) | | |
| Low-density lipoprotein receptor-related | LRP10 MSTP087 | SEQ ID NO: 24 |
| protein 10 (LRP-10) | SP220 | |
| | UNQ389/PRO724 | |
| Receptor-type tyrosine-protein | PTPRC CD45 | SEQ ID NO: 25 |
| phosphatase C (EC 3.1.3.48) (Leukocyte | | |
| common antigen) (L-CA) (T200) (CD antigen | | |
| CD45) | | |
| Cytokine receptor common subunit gamma | IL2RG | SEQ ID NO: 26 |
| (Interleukin-2 receptor subunit gamma) (IL- | | |
| 2 receptor subunit gamma) (IL-2R subunit | | |
| gamma) (IL-2RG) (gammaC) (p64) (CD | | |
| antigen CD132) | | |
| Tumor necrosis factor receptor superfamily | TNFRSF17 BCM | SEQ ID NO: 27 |
| member 17 (B-cell maturation protein) (CD | BCMA | |
| antigen CD269) | | |
| Tumor necrosis factor receptor superfamily | TNFRSF10A APO2 | SEQ ID NO: 28 |
| member 10A (Death receptor 4) (TNF- | DR4 TRAILR1 | |
| related apoptosis-inducing ligand receptor | | |
| 1) (TRAIL receptor 1) (TRAIL-R1) (CD | | |
| antigen CD261) | | |
| SLAM family member 7 (CD2 subset 1) | SLAMF7 CS1 | SEQ ID NO: 29 |
| (CD2-like receptor-activating cytotoxic | UNQ576/PRO1138 | |
| cells) (CRACC) (Membrane protein FOAP- | | |
| 12) (Novel Ly9) (Protein 19A) (CD antigen | | |
| CD319) | | |
| Tumor necrosis factor receptor superfamily | TNFRSF1B TNFBR | SEQ ID NO: 30 |
| member 1B (Tumor necrosis factor | TNFR2 | |
| receptor 2) (TNF-R2) (Tumor necrosis factor | | |
| receptor type II) (TNF-RII) (TNFR-II) (p75) | | |
| (p80 TNF-alpha receptor) (CD antigen | | |
| CD120b) (Etanercept) [Cleaved into: Tumor | | |
| necrosis factor receptor superfamily | | |
| member 1b, membrane form; Tumor | | |
| necrosis factor-binding protein 2 (TBP-2) | | |
| (TBPII)] | | |
| Leukocyte-associated immunoglobulin-like | LAIR1 CD305 | SEQ ID NO: 31 |
| receptor 1 (LAIR-1) (hLAIR1) (CD antigen | | |
| CD305) | | |
| Toll-like receptor 1 (EC 3.2.2.6) | TLR1 KIAA0012 | SEQ ID NO: 32 |
| (Toll/interleukin-1 receptor-like protein) | | |
| (TIL) (CD antigen CD281) | | |
| Toll-like receptor 7 | TLR7 | SEQ ID NO: 33 |
| | UNQ248/PRO285 | |
| Tumor necrosis factor receptor superfamily | TNFRSF18 AITR GITR | SEQ ID NO: 34 |
| member 18 (Activation-inducible TNFR | UNQ319/PRO364 | |
| family receptor) (Glucocorticoid-induced | | |
| TNFR-related protein) (CD antigen CD357) | | |
| Tumor necrosis factor receptor superfamily | TNFRSF8 CD30 | SEQ ID NO: 35 |
| member 8 (CD30L receptor) (Ki-1 antigen) | D1S166E | |
| (Lymphocyte activation antigen CD30) (CD | | |
| antigen CD30) | | |
| Interferon alpha/beta receptor 2 (IFN-R-2) | IFNAR2 IFNABR | SEQ ID NO: 36 |
| (IFN-alpha binding protein) (IFN-alpha/beta | IFNARB | |
| receptor 2) (Interferon alpha binding | | |
| protein) (Type I interferon receptor 2) | | |
| SLAM family member 6 (Activating NK | SLAMF6 KALI | SEQ ID NO: 37 |
| receptor) (NK-T-B-antigen) (NTB-A) (CD | UNQ6123/PRO20080 | |
| antigen CD352) | | |
| Tumor necrosis factor receptor superfamily | TNFRSF10D DCR2 | SEQ ID NO: 38 |
| member 10D (Decoy receptor 2) (DcR2) | TRAILR4 TRUNDD | |
| (TNF-related apoptosis-inducing ligand | UNQ251/PRO288 | |
| receptor 4) (TRAIL receptor 4) (TRAIL-R4) | | |
| (TRAIL receptor with a truncated death | | |
| domain) (CD antigen CD264) | | |
| P-selectin glycoprotein ligand 1 (PSGL-1) | SELPLG | SEQ ID NO: 39 |
| (Selectin P ligand) (CD antigen CD162) | | |
| T-cell surface protein tactile (Cell surface | CD96 | SEQ ID NO: 40 |
| antigen CD96) (T cell-activated increased | | |
| late expression protein) (CD antigen CD96) | | |
| Porimin (Keratinocytes-associated | TMEM123 KCT3 | SEQ ID NO: 41 |
| transmembrane protein 3) (KCT-3) (Pro- | PSEC0111 | |
| oncosis receptor inducing membrane | UNQ641/PRO1271 | |
| injury) (Transmembrane protein 123) | | |
| Integrin alpha-4 (CD49 antigen-like family | ITGA4 CD49D | SEQ ID NO: 42 |
| member D) (Integrin alpha-IV) (VLA-4 | | |
| subunit alpha) (CD antigen CD49d) | | |
| G-protein coupled receptor 15 (Brother of | GPR15 | SEQ ID NO: 43 |
| Bonzo) (BoB) | | |
| Multidrug resistance-associated protein 1 | ABCC1 MRP MRP1 | SEQ ID NO: 44 |
| (EC 7.6.2.2) (ATP-binding cassette sub- | | |
| family C member 1) (Glutathione-S- | | |
| conjugate-translocating ATPase ABCC1) (EC | | |
| 7.6.2.3) (Leukotriene C(4) transporter) | | |
| (LTC4 transporter) | | |
| CX3C chemokine receptor 1 (C-X3-C CKR-1) | CX3CR1 CMKBRL1 | SEQ ID NO: 45 |
| (CX3CR1) (Beta chemokine receptor-like 1) | GPR13 | |
| (CMK-BRL-1) (CMK-BRL1) (Fractalkine | | |
| receptor) (G-protein coupled receptor 13) | | |
| (V28) | | |
| B-lymphocyte antigen CD20 (B-lymphocyte | MS4A1 CD20 | SEQ ID NO: 46 |
| surface antigen B1) (Bp35) (Leukocyte | | |
| surface antigen Leu-16) (Membrane- | | |
| spanning 4-domains subfamily A member | | |
| 1) (CD antigen CD20) | | |
| HLA class II histocompatibility antigen, DM | HLA-DMB DMB | SEQ ID NO: 47 |
| beta chain (MHC class II antigen DMB) | RING7 | |
| (Really interesting new gene 7 protein) | | |
| Low affinity immunoglobulin gamma Fc | FCGR2B CD32 FCG2 | SEQ ID NO: 48 |
| region receptor II-b (IgG Fc receptor II-b) | IGFR2 | |
| (CDw32) (Fc-gamma RII-b) (Fc-gamma-RIIb) | | |
| (FcRII-b) (CD antigen CD32) | | |
| HLA class I histocompatibility antigen, | HLA-F HLA-5.4 HLAF | SEQ ID NO: 49 |
| alpha chain F (CDA12) (HLA F antigen) | | |
| (Leukocyte antigen F) (MHC class I antigen | | |
| F) | | |
| Disintegrin and metalloproteinase domain- | ADAM9 KIAA0021 | SEQ ID NO: 50 |
| containing protein 9 (ADAM 9) (EC 3.4.24.-) | MCMP MDC9 | |
| (Cellular disintegrin-related protein) | MLTNG | |
| (Meltrin-gamma) | | |
| (Metalloprotease/disintegrin/cysteine-rich | | |
| protein 9) (Myeloma cell | | |
| metalloproteinase) | | |
| IgG receptor FcRn large subunit p51 (FcRn) | FCGRT FCRN | SEQ ID NO: 51 |
| (IgG Fc fragment receptor transporter | | |
| alpha chain) (Neonatal Fc receptor) | | |
| Interleukin-6 receptor subunit beta (IL-6 | IL6ST | SEQ ID NO: 52 |
| receptor subunit beta) (IL-6R subunit beta) | | |
| (IL-6R-beta) (IL-6RB) (CDw130) (Interleukin- | | |
| 6 signal transducer) (Membrane | | |
| glycoprotein 130) (gp130) (Oncostatin-M | | |
| receptor subunit alpha) (CD antigen CD130) | | |
| Lymphocyte function-associated antigen 3 | CD58 LFA3 | SEQ ID NO: 53 |
| (Ag3) (Surface glycoprotein LFA-3) (CD | | |
| antigen CD58) | | |
| C-C chemokine receptor type 10 (C-C CKR- | CCR10 GPR2 | SEQ ID NO: 54 |
| 10) (CC-CKR-10) (CCR-10) (G-protein | | |
| coupled receptor 2) | | |
| C-type lectin domain family 7 member A | CLEC7A BGR | SEQ ID NO: 55 |
| (Beta-glucan receptor) (C-type lectin | CLECSF12 DECTIN1 | |
| superfamily member 12) (Dendritic cell- | UNQ539/PRO1082 | |
| associated C-type lectin 1) (DC-associated | | |
| C-type lectin 1) (Dectin-1) (CD antigen | | |
| CD369) | | |
| CD320 antigen (8D6 antigen) (FDC-signaling | CD320 8D6A | SEQ ID NO: 56 |
| molecule 8D6) (FDC-SM-8D6) | UNQ198/PRO224 | |
| (Transcobalamin receptor) (TCblR) (CD | | |
| antigen CD320) | | |
| Fc receptor-like protein 3 (FcR-like protein | FCRL3 FCRH3 IFGP3 | SEQ ID NO: 57 |
| 3) (FcRL3) (Fc receptor homolog 3) (FcRH3) | IRTA3 SPAP2 | |
| (IFGP family protein 3) (hIFGP3) (Immune | | |
| receptor translocation-associated protein | | |
| 3) (SH2 domain-containing phosphatase | | |
| anchor protein 2) (CD antigen CD307c) | | |
| Plexin-C1 (Virus-encoded semaphorin | PLXNC1 VESPR | SEQ ID NO: 58 |
| protein receptor) (CD antigen CD232) | | |
| Interleukin-17 receptor A (IL-17 receptor A) | IL17RA IL17R | SEQ ID NO: 59 |
| (IL-17RA) (CDw217) (CD antigen CD217) | | |
| C-C chemokine receptor type 1 (C-C CKR-1) | CCR1 CMKBR1 | SEQ ID NO: 60 |
| (CC-CKR-1) (CCR-1) (CCR1) (HM145) (LD78 | CMKR1 SCYAR1 | |
| receptor) (Macrophage inflammatory | | |
| protein 1-alpha receptor) (MIP-1alpha-R) | | |
| (RANTES-R) (CD antigen CD191) | | |
| T-lymphocyte activation antigen CD80 | CD80 CD28LG | SEQ ID NO: 61 |
| (Activation B7-1 antigen) (BB1) (CTLA-4 | CD28LG1 LAB7 | |
| counter-receptor B7.1) (B7) (CD antigen | | |
| CD80) | | |
| Proteinase-activated receptor 4 (PAR-4) | F2RL3 PAR4 | SEQ ID NO: 62 |
| (Coagulation factor II receptor-like 3) | | |
| (Thrombin receptor-like 3) | | |
| Interleukin-6 receptor subunit alpha (IL-6 | IL6R | SEQ ID NO: 63 |
| receptor subunit alpha) (IL-6R subunit | | |
| alpha) (IL-6R-alpha) (IL-6RA) (IL-6R 1) | | |
| (Membrane glycoprotein 80) (gp80) (CD | | |
| antigen CD126) | | |
| Hepatitis A virus cellular receptor 2 (HAVcr- | HAVCR2 TIM3 TIMD3 | SEQ ID NO: 64 |
| 2) (T-cell immunoglobulin and mucin | | |
| domain-containing protein 3) (TIMD-3) (T- | | |
| cell immunoglobulin mucin receptor 3) | | |
| (TIM-3) (T-cell membrane protein 3) (CD | | |
| antigen CD366) | | |
| Interleukin-27 receptor subunit alpha (IL-27 | IL27RA CRL1 TCCR | SEQ ID NO: 65 |
| receptor subunit alpha) (IL-27R subunit | WSX1 | |
| alpha) (IL-27R-alpha) (IL-27RA) (Cytokine | UNQ296/PRO336 | |
| receptor WSX-1) (Cytokine receptor-like 1) | | |
| (Type I T-cell cytokine receptor) (TCCR) | | |
| (ZcytoR1) | | |
| Interleukin-13 receptor subunit alpha-2 (IL- | IL13RA2 IL13R | SEQ ID NO: 66 |
| 13 receptor subunit alpha-2) (IL-13R | | |
| subunit alpha-2) (IL-13R-alpha-2) (IL- | | |
| 13RA2) (Interleukin-13-binding protein) (CD | | |
| antigen CD213a2) | | |
| Integrin beta-7 (Gut homing receptor beta | ITGB7 | SEQ ID NO: 67 |
| subunit) | | |
| Fc receptor-like protein 2 (FcR-like protein | FCRL2 FCRH2 IFGP4 | SEQ ID NO: 68 |
| 2) (FcRL2) (Fc receptor homolog 2) (FcRH2) | IRTA4 SPAP1 | |
| (IFGP family protein 4) (Immunoglobulin | UNQ9236/PRO31998 | |
| receptor translocation-associated protein | | |
| 4) (SH2 domain-containing phosphatase | | |
| anchor protein 1) (CD antigen CD307b) | | |
| L-selectin (CD62 antigen-like family | SELL LNHR LYAM1 | SEQ ID NO: 69 |
| member L) (Leukocyte adhesion molecule | | |
| 1) (LAM-1) (Leukocyte surface antigen Leu- | | |
| 8) (Leukocyte-endothelial cell adhesion | | |
| molecule 1) (LECAM1) (Lymph node | | |
| homing receptor) (TQ1) (gp90-MEL) (CD | | |
| antigen CD62L) | | |
| Intercellular adhesion molecule 2 (ICAM-2) | ICAM2 | SEQ ID NO: 70 |
| (CD antigen CD102) | | |
| CD82 antigen (C33 antigen) (IA4) (Inducible | CD82 KAI1 SAR2 ST6 | SEQ ID NO: 71 |
| membrane protein R2) (Metastasis | TSPAN27 | |
| suppressor Kangai-1) (Suppressor of | | |
| tumorigenicity 6 protein) (Tetraspanin-27) | | |
| (Tspan-27) (CD antigen CD82) | | |
| CD83 antigen (hCD83) (B-cell activation | CD83 | SEQ ID NO: 72 |
| protein) (Cell surface protein HB15) (CD | | |
| antigen CD83) | | |
| Activin receptor type-1B (EC 2.7.11.30) | ACVR1B ACVRLK4 | SEQ ID NO: 73 |
| (Activin receptor type IB) (ACTR-IB) (Activin | ALK4 | |
| receptor-like kinase 4) (ALK-4) | | |
| (Serine/threonine-protein kinase receptor | | |
| R2) (SKR2) | | |
| Lymphocyte antigen 75 (Ly-75) (C-type | LY75 CD205 CLEC13B | SEQ ID NO: 74 |
| lectin domain family 13 member B) (DEC- | | |
| 205) (gp200-MR6) (CD antigen CD205) | | |
| Junctional adhesion molecule B (JAM-B) | JAM2 C21orf43 | SEQ ID NO: 75 |
| (Junctional adhesion molecule 2) (JAM-2) | VEJAM | |
| (Vascular endothelial junction-associated | UNQ219/PRO245 | |
| molecule) (VE-JAM) (CD antigen CD322) | | |
| Low-density lipoprotein receptor-related | LRP8 APOER2 | SEQ ID NO: 76 |
| protein 8 (LRP-8) (Apolipoprotein E | | |
| receptor 2) | | |
| Cytokine receptor common subunit beta | CSF2RB IL3RB IL5RB | SEQ ID NO: 77 |
| (CDw131) (GM-CSF/IL-3/IL-5 receptor | | |
| common beta subunit) (CD antigen CD131) | | |
| Interleukin-10 receptor subunit beta (IL-10 | IL10RB CRFB4 | SEQ ID NO: 78 |
| receptor subunit beta) (IL-10R subunit | D21S58 D21S66 | |
| beta) (IL-10RB) (Cytokine receptor class-II | | |
| member 4) (Cytokine receptor family 2 | | |
| member 4) (CRF2-4) (Interleukin-10 | | |
| receptor subunit 2) (IL-10R subunit 2) (IL- | | |
| 10R2) (CD antigen CDw210b) | | |
| Interferon gamma receptor 1 (IFN-gamma | IFNGR1 | SEQ ID NO: 79 |
| receptor 1) (IFN-gamma-R1) (CDw119) | | |
| (Interferon gamma receptor alpha-chain) | | |
| (IFN-gamma-R-alpha) (CD antigen CD119) | | |
| Adhesion G-protein coupled receptor G6 | ADGRG6 DREG | SEQ ID NO: 80 |
| (Developmentally regulated G-protein- | GPR126 VIGR | |
| coupled receptor) (G-protein coupled | | |
| receptor 126) (Vascular inducible G | | |
| protein-coupled receptor) [Cleaved into: | | |
| ADGRG6 N-terminal fragment (ADGRG6- | | |
| NTF); ADGRG6 C-terminal fragment | | |
| (ADGRG6-CTF)] | | |
| Adenosine receptor A2a | ADORA2A ADORA2 | SEQ ID NO: 81 |
| Progressive ankylosis protein homolog | ANKH KIAA1581 | SEQ ID NO: 82 |
| (ANK) | UNQ241/PRO274 | |
| Cell adhesion molecule-related/down- | CDON CDO | SEQ ID NO: 83 |
| regulated by oncogenes | | |
| CSC1-like protein 1 (Transmembrane | TMEM63A KIAA0489 | SEQ ID NO: 84 |
| protein 63A) | KIAA0792 | |
| Toll-like receptor 9 (CD antigen CD289) | TLR9 | SEQ ID NO: 85 |
| | UNQ5798/PRO19605 | |
| Sodium- and chloride-dependent taurine | SLC6A6 | SEQ ID NO: 86 |
| transporter (Solute carrier family 6 | | |
| member 6) | | |
| CD48 antigen (B-lymphocyte activation | CD48 BCM1 BLAST1 | SEQ ID NO: 87 |
| marker BLAST-1) (BCM1 surface antigen) | | |
| (Leukocyte antigen MEM-102) (SLAM | | |
| family member 2) (SLAMF2) (Signaling | | |
| lymphocytic activation molecule 2) (TCT.1) | | |
| (CD antigen CD48) | | |
| ADP-ribosyl cyclase/cyclic ADP-ribose | CD38 | SEQ ID NO: 88 |
| hydrolase 1 (EC 3.2.2.6) (2′-phospho-ADP- | | |
| ribosyl cyclase) (2′-phospho-ADP-ribosyl | | |
| cyclase/2′-phospho-cyclic-ADP-ribose | | |
| transferase) (EC 2.4.99.20) (2′-phospho- | | |
| cyclic-ADP-ribose transferase) (ADP-ribosyl | | |
| cyclase 1) (ADPRC 1) (Cyclic ADP-ribose | | |
| hydrolase 1) (cADPr hydrolase 1) (T10) (CD | | |
| antigen CD38) | | |
| Muscarinic acetylcholine receptor M3 | CHRM3 | SEQ ID NO: 89 |
| Macrophage colony-stimulating factor 1 | CSF1 | SEQ ID NO: 90 |
| (CSF-1) (M-CSF) (MCSF) (Lanimostim) | | |
| [Cleaved into: Processed macrophage | | |
| colony-stimulating factor 1] | | |
| Multidrug resistance-associated protein 4 | ABCC4 MRP4 | SEQ ID NO: 91 |
| (ATP-binding cassette sub-family C member | | |
| 4) (MRP/cMOAT-related ABC transporter) | | |
| (Multi-specific organic anion transporter B) | | |
| (MOAT-B) | | |
| Potassium voltage-gated channel subfamily | KCNA3 HGK5 | SEQ ID NO: 92 |
| A member 3 (HGK5) (HLK3) (HPCN3) | | |
| (Voltage-gated K(+) channel HuKIII) | | |
| (Voltage-gated potassium channel subunit | | |
| Kv1.3) | | |
| Sialic acid-binding Ig-like lectin 7 (Siglec-7) | SIGLEC7 AIRM1 | SEQ ID NO: 93 |
| (Adhesion inhibitory receptor molecule 1) | | |
| (AIRM-1) (CDw328) (D-siglec) (QA79 | | |
| membrane protein) (p75) (CD antigen | | |
| CD328) | | |
| Sphingosine 1-phosphate receptor 4 (S1P | S1PR4 EDG6 | SEQ ID NO: 94 |
| receptor 4) (S1P4) (Endothelial | | |
| differentiation G-protein coupled receptor | | |
| 6) (Sphingosine 1-phosphate receptor Edg- | | |
| 6) (S1P receptor Edg-6) | | |
| Transmembrane protein 106B | TMEM106B | SEQ ID NO: 95 |
| Proton-coupled folate transporter (G21) | SLC46A1 HCP1 PCFT | SEQ ID NO: 96 |
| (Heme carrier protein 1) (PCFT/HCP1) | | |
| (Solute carrier family 46 member 1) | | |
| Allergin-1 (Allergy inhibitory receptor 1) | MILR1 C17orf60 | SEQ ID NO: 97 |
| (Mast cell antigen 32) (MCA-32) (Mast cell | MCA32 | |
| immunoglobulin-like receptor 1) | | |
| Steryl-sulfatase (EC 3.1.6.2) (Arylsulfatase | STS ARSC1 | SEQ ID NO: 98 |
| C) (ASC) (Estrone sulfatase) (Steroid | | |
| sulfatase) (Steryl-sulfate sulfohydrolase) | | |
| Tumor necrosis factor receptor superfamily | FAS APT1 FAS1 | SEQ ID NO: 99 |
| member 6 (Apo-1 antigen) (Apoptosis- | TNFRSF6 | |
| mediating surface antigen FAS) (FASLG | | |
| receptor) (CD antigen CD95) | | |
| Presenilin-2 (PS-2) (EC 3.4.23.-) (AD3LP) | PSEN2 AD4 PS2 | SEQ ID NO: 100 |
| (AD5) (E5-1) (STM-2) [Cleaved into: | PSNL2 STM2 | |
| Presenilin-2 NTF subunit; Presenilin-2 CTF | | |
| subunit] | | |
| Solute carrier organic anion transporter | SLCO4A1 OATP1 | SEQ ID NO: 101 |
| family member 4A1 (OATP4A1) (Colon | OATP4A1 OATPE | |
| organic anion transporter) (Organic anion | SLC21A12 | |
| transporter polypeptide-related protein 1) | | |
| (OATP-RP1) (OATPRP1) (POAT) (Organic | | |
| anion-transporting polypeptide E) (OATP-E) | | |
| (Sodium-independent organic anion | | |
| transporter E) (Solute carrier family 21 | | |
| member 12) | | |
| Solute carrier organic anion transporter | SLCO3A1 OATP3A1 | SEQ ID NO: 102 |
| family member 3A1 (OATP3A1) (Organic | OATPD SLC21A11 | |
| anion transporter polypeptide-related | | |
| protein 3) (OATP-RP3) (OATPRP3) (Organic | | |
| anion-transporting polypeptide D) (OATP- | | |
| D) (PGE1 transporter) (Sodium- | | |
| independent organic anion transporter D) | | |
| (Solute carrier family 21 member 11) | | |
| Transmembrane protein 154 | TMEM154 | SEQ ID NO: 103 |
| Semaphorin-4A (Semaphorin-B) (Sema B) | SEMA4A SEMAB | SEQ ID NO: 104 |
| | SEMB | |
| | UNQ783/PRO1317 | |
| Multidrug resistance-associated protein 5 | ABCC5 MRP5 | SEQ ID NO: 105 |
| (ATP-binding cassette sub-family C member | | |
| 5) (Multi-specific organic anion transporter | | |
| C) (MOAT-C) (SMRP) (pABC11) | | |
| Transmembrane protein 132E | TMEM132E | SEQ ID NO: 106 |
| Netrin receptor UNC5C (Protein unc-5 | UNC5C UNC5H3 | SEQ ID NO: 107 |
| homolog 3) (Protein unc-5 homolog C) | | |
| Scavenger receptor class B member 1 | SCARB1 CD36L1 | SEQ ID NO: 108 |
| (SRB1) (CD36 and LIMPII analogous 1) (CLA- | CLA1 | |
| 1) (CD36 antigen-like 1) (Collagen type I | | |
| receptor, thrombospondin receptor-like 1) | | |
| (SR-BI) (CD antigen CD36) | | |
| Zinc transporter ZIP6 (Estrogen-regulated | SLC39A6 LIV1 ZIP6 | SEQ ID NO: 109 |
| protein LIV-1) (Solute carrier family 39 | | |
| member 6) (Zrt- and Irt-like protein 6) (ZIP- | | |
| 6) | | |
| Inactive tyrosine-protein kinase | ROR1 NTRKR1 | SEQ ID NO: 110 |
| transmembrane receptor ROR1 | | |
| (Neurotrophic tyrosine kinase, receptor- | | |
| related 1) | | |
| Solute carrier family 26 member 6 (Anion | SLC26A6 | SEQ ID NO: 111 |
| exchange transporter) (Pendrin-like protein | | |
| 1) (Pendrin-L1) | | |
| SLIT and NTRK-like protein 5 (Leucine-rich | SLITRK5 KIAA0918 | SEQ ID NO: 112 |
| repeat-containing protein 11) | LRRC11 | |
| Cell surface glycoprotein CD200 receptor 1 | CD200R1 CD200R | SEQ ID NO: 113 |
| (CD200 cell surface glycoprotein receptor) | CRTR2 MOX2R OX2R | |
| (Cell surface glycoprotein OX2 receptor 1) | UNQ2522/PRO6015 | |
| Discoidin, CUB and LCCL domain-containing | DCBLD1 | SEQ ID NO: 114 |
| protein 1 | | |
| CD109 antigen (150 kDa TGF-beta-1- | CD109 CPAMD7 | SEQ ID NO: 115 |
| binding protein) (C3 and PZP-like alpha-2- | | |
| macroglobulin domain-containing protein | | |
| 7) (Platelet-specific Gov antigen) (p180) | | |
| (r150) (CD antigen CD109) | | |
| C3a anaphylatoxin chemotactic receptor | C3AR1 AZ3B C3R1 | SEQ ID NO: 116 |
| (C3AR) (C3a-R) | HNFAG09 | |
| CD70 antigen (CD27 ligand) (CD27-L) | CD70 CD27L CD27LG | SEQ ID NO: 117 |
| (Tumor necrosis factor ligand superfamily | TNFSF7 | |
| member 7) (CD antigen CD70) | | |
| Large neutral amino acids transporter small | SLC43A2 LAT4 | SEQ ID NO: 118 |
| subunit 4 (L-type amino acid transporter 4) | PP7664 | |
| (Solute carrier family 43 member 2) | | |
| HLA class II histocompatibility antigen, DR | HLA-DRB5 | SEQ ID NO: 119 |
| beta 5 chain (DR beta-5) (DR2-beta-2) | | |
| (Dw2) (MHC class II antigen DRB5) | | |
| Ephrin type-A receptor 5 (EC 2.7.10.1) | EPHA5 BSK EHK1 | SEQ ID NO: 120 |
| (Brain-specific kinase) (EPH homology | HEK7 TYRO4 | |
| kinase 1) (EHK-1) (EPH-like kinase 7) (EK7) | | |
| (hEK7) | | |
| Monocarboxylate transporter 2 (MCT 2) | SLC16A7 MCT2 | SEQ ID NO: 121 |
| (Solute carrier family 16 member 7) | | |
| Cysteine-rich motor neuron 1 protein | CRIM1 S52 | SEQ ID NO: 122 |
| (CRIM-1) (Cysteine-rich repeat-containing | UNQ1886/PRO4330 | |
| protein S52) [Cleaved into: Processed | | |
| cysteine-rich motor neuron 1 protein] | | |
| Nuclear envelope integral membrane | NEMP1 KIAA0286 | SEQ ID NO: 123 |
| protein 1 | TMEM194 | |
| | TMEM194A | |
| HLA class II histocompatibility antigen, DR | HLA-DRB4 | SEQ ID NO: 124 |
| beta 4 chain (MHC class II antigen DRB4) | | |
| Cationic amino acid transporter 2 (CAT-2) | SLC7A2 ATRC2 CAT2 | SEQ ID NO: 125 |
| (CAT2) (Low affinity cationic amino acid | | |
| transporter 2) (Solute carrier family 7 | | |
| member 2) | | |
| Integral membrane protein 2C (Cerebral | ITM2C BRI3 hucep- | SEQ ID NO: 126 |
| protein 14) (Transmembrane protein BRI3) | 14 NPD018 | |
| [Cleaved into: CT-BRI3] | PSEC0047 | |
| Ephrin type-A receptor 3 (EC 2.7.10.1) | EPHA3 ETK ETK1 HEK | SEQ ID NO: 127 |
| (EPH-like kinase 4) (EK4) (hEK4) (HEK) | TYRO4 | |
| (Human embryo kinase) (Tyrosine-protein | | |
| kinase TYRO4) (Tyrosine-protein kinase | | |
| receptor ETK1) (Eph-like tyrosine kinase 1) | | |
| Lysosome-associated membrane | LAMP5 C20orf103 | SEQ ID NO: 128 |
| glycoprotein 5 (Brain and dendritic cell- | | |
| associated LAMP) (Brain-associated LAMP- | | |
| like protein) (BAD-LAMP) (Lysosome- | | |
| associated membrane protein 5) (LAMP-5) | | |
| Metal transporter CNNM2 (Ancient | CNNM2 ACDP2 | SEQ ID NO: 129 |
| conserved domain-containing protein 2) | | |
| (Cyclin-M2) | | |
| CD177 antigen (Human neutrophil | CD177 NB1 PRV1 | SEQ ID NO: 130 |
| alloantigen 2a) (HNA-2a) (NB1 | UNQ595/PRO1181 | |
| glycoprotein) (NB1 GP) (Polycythemia rubra | | |
| vera protein 1) (PRV-1) (CD antigen CD177) | | |
| Zinc transporter ZIP10 (Solute carrier family | SLC39A10 KIAA1265 | SEQ ID NO: 131 |
| 39 member 10) (Zrt- and Irt-like protein 10) | ZIP10 | |
| (ZIP-10) | | |
| Plexin-A3 (Plexin-4) (Semaphorin receptor | PLXNA3 PLXN4 SEX | SEQ ID NO: 132 |
| SEX) | | |
| Phospholipid phosphatase 2 (EC 3.1.3.-) (EC | PLPP2 LPP2 PPAP2C | SEQ ID NO: 133 |
| 3.1.3.4) (Lipid phosphate | | |
| phosphohydrolase 2) (PAP2-gamma) | | |
| (PAP2-G) (Phosphatidate | | |
| phosphohydrolase type 2c) (Phosphatidic | | |
| acid phosphatase 2c) (PAP-2c) (PAP2c) | | |
| Serine incorporator 3 (Tumor differentially | SERINC3 DIFF33 | SEQ ID NO: 134 |
| expressed protein 1) | TDE1 SBBI99 | |
| Sodium/myo-inositol cotransporter | SLC5A3 | SEQ ID NO: 135 |
| (Na(+)/myo-inositol cotransporter) | | |
| (Sodium/myo-inositol transporter 1) | | |
| (SMIT1) (Solute carrier family 5 member 3) | | |
| Teneurin-3 (Ten-3) (Protein Odd Oz/ten-m | TENM3 KIAA1455 | SEQ ID NO: 136 |
| homolog 3) (Tenascin-M3) (Ten-m3) | ODZ3 TNM3 | |
| (Teneurin transmembrane protein 3) | | |
| Sodium-coupled neutral amino acid | SLC38A5 JM24 SN2 | SEQ ID NO: 137 |
| transporter 5 (Solute carrier family 38 | SNAT5 PP7194 | |
| member 5) (System N transporter 2) | | |
| Solute carrier family 23 member 2 (Na(+)/L- | SLC23A2 KIAA0238 | SEQ ID NO: 138 |
| ascorbic acid transporter 2) (Nucleobase | NBTL1 SLC23A1 | |
| transporter-like 1 protein) (Sodium- | SVCT2 YSPL2 | |
| dependent vitamin C transporter 2) (hSVCT2) | | |
| (Yolk sac permease-like molecule 2) | | |
| Sialin (H(+)/nitrate cotransporter) | SLC17A5 | SEQ ID NO: 139 |
| (H(+)/sialic acid cotransporter) (AST) | | |
| (Membrane glycoprotein HP59) (Solute | | |
| carrier family 17 member 5) (Vesicular | | |
| H(+)/Aspartate-glutamate cotransporter) | | |
| Urokinase plasminogen activator surface | PLAUR MO3 UPAR | SEQ ID NO: 140 |
| receptor (U-PAR) (uPAR) (Monocyte | | |
| activation antigen Mo3) (CD antigen CD87) | | |
| Sn1-specific diacylglycerol lipase beta (DGL- | DAGLB | SEQ ID NO: 141 |
| beta) (EC 3.1.1.-) (KCCR13L) | | |
| Embigin | EMB | SEQ ID NO: 142 |
| Protein HEG homolog 1 | HEG1 KIAA1237 | SEQ ID NO: 143 |
| Plexin-A4 | PLXNA4 KIAA1550 | SEQ ID NO: 144 |
| | PLXNA4A PLXNA4B | |
| | UNQ2820/PRO34003 | |
| Protein ELFN1 (Extracellular leucine-rich | ELFN1 PPP1R28 | SEQ ID NO: 145 |
| repeat and fibronectin type-III domain- | | |
| containing protein 1) (Protein phosphatase | | |
| 1 regulatory subunit 28) | | |
| Adhesion G-protein coupled receptor G5 | ADGRG5 GPR114 | SEQ ID NO: 146 |
| (G-protein coupled receptor 114) (G- | PGR27 | |
| protein coupled receptor PGR27) | UNQ2524/PRO6017 | |
| Leucine-rich repeat and fibronectin type III | LRFN1 KIAA1484 | SEQ ID NO: 147 |
| domain-containing protein 1 (Synaptic | SALM2 | |
| adhesion-like molecule 2) | | |
| CD160 antigen (Natural killer cell receptor | CD160 BY55 | SEQ ID NO: 148 |
| BY55) (CD antigen CD160) [Cleaved into: | | |
| CD160 antigen, soluble form] | | |
| Battenin (Batten disease protein) (Protein | CLN3 BTS | SEQ ID NO: 149 |
| CLN3) | | |
| Transmembrane protein 178B | TMEM178B | SEQ ID NO: 150 |
| RGM domain family member B (DRG11- | RGMB | SEQ ID NO: 151 |
| responsive axonal guidance and outgrowth | | |
| of neurite) (DRAGON) | | |
| Reversion-inducing cysteine-rich protein | RECK ST15 | SEQ ID NO: 152 |
| with Kazal motifs (hRECK) (Suppressor of | | |
| tumorigenicity 15 protein) | | |
| Natural cytotoxicity triggering receptor 3 | NCR3LG1 B7H6 | SEQ ID NO: 153 |
| ligand 1 (B7 homolog 6) (B7-H6) | | |
| Testisin (EC 3.4.21.-) (Eosinophil serine | PRSS21 ESP1 TEST1 | SEQ ID NO: 154 |
| protease 1) (ESP-1) (Serine protease 21) | UNQ266/PRO303 | |
| UL16-binding protein 3 (ALCAN-gamma) | ULBP3 N2DL3 | SEQ ID NO: 155 |
| (NKG2D ligand 3) (N2DL-3) (NKG2DL3) | RAET1N | |
| (Retinoic acid early transcript 1N) | | |

### Example 2

### Additional MM Surface Protein Analysis

The unbiased pool of 5,454 Uniprot IDs corresponding to 4,761 proteins identified in the surface-specific proteomic analyses of seven multiple myeloma cell lines as described in Example 1 was subjected to further analysis.

Given the lack of bioinformatics tools enabling accurate detection of proteins at the cell surface location, we developed an integrated scoring database for surface protein annotation. To this purpose we merged five published databases using different methodologies in identifying molecules that localized to the plasma membrane: #1 (Dìaz-Ramos et al, Immunol Lett 2011, 134, 183-187, doi:10.1016/j.imlet.2010.09.016) was manually curated from the literature; #2 (Baush-Fluck et al. Proc Natl Acad Sci USA 2018, 115, E10988-E10997, doi:10.1073/pnas.1808790115.) was computationally compiled using a random forest classifier trained on domain-specific protein features and tested on a set of α-helical transmembrane proteins; #3 (Town et al. Proc Natl Acad Sci USA 2016, 113, 3603-3608, doi:10.1073/pnas.1521251113) was computationally compiled using GO and Uniprot annotation followed by transmembrane domain prediction and signal peptide prediction; #4 (da Cunha et al. Proc Natl Acad Sci USA 2009, 106, 16752-16757, doi:10.1073/pnas.0907939106) was computationally compiled by transmembrane domain prediction; #5 (Thule et al.) was experimentally determined by antibody-based immunofluorescence microscopy. In our integrative computational database, each uniprot ID is scored based on the number of databases it was identified in, with 0 denoting the protein was not found in any and 5 denoting the protein was found in all five (FIG. 1B). Based on this integrative computational tool cutoff 1 includes 1229 uniprot IDs, cutoff 2: 699 uniprot IDs, cutoff 3: 448 uniprot IDs, cutoff 4: 260 uniprot IDs and cutoff 5: 63 uniprot IDs. For further analyses we selected proteins with a surface score equal or higher than three representing a high-level of confidence for cell surface location.

We integrated the CoMMpass RNA-seq dataset providing gene expression data from 904 MM patients. This integrative approach served to identify surface targets that are relevant to the patient population. We applied our surface scoring system to 16,462 transcripts from MM patients and, identified 402 targets with a surface score higher than three and detected by MS (FIG. 1C). We have further selected 326 top surface proteins overlapping with high expressors in patients by calculating 1SD from the mean gene expression in patients. We calculated the average expression of each gene and removed low expressing genes (1 SD below the mean) and selected the genes that have expression over the cutoff (326 transcripts kept) (FIG. 1D). Of note, patient gene expression was unimodal except for 7 transcripts presenting a non-unimodal expression (NCAM1, TPBG, ROB1, LAMP5, APP, PTRCAP, PLXAN2).

This group of 326 surface proteins includes known MM-associated surface proteins, some of which are targeted in clinical and pre-clinical studies such as BCMA, SLAMF7, TACI, LY9, CD38. GPCR5D and FCRL5, also currently investigated in clinical trials for patients with MM did not result in this list because they were identified by transcriptomic analysis in patients, but we did not detect their protein expression by MS analysis.

### The MM Surfaceome Mainly Consists in Immune-Related Proteins

In order to gain insights into the function of these candidate surface proteins we performed functional enrichment analyses and, found that they can be divided into three main functional clusters: 1) proteins involved in immune-mediated pathways 2) transporters 3) proteins mediating the adhesion to the stroma. The surface proteins related to immune-mediated pathways (top cluster) involve 227 out of 326 molecules and thus, might mediate the interplay between immune cells and myeloma cells.

By further digging into this group of surface proteins with the Kegg and Reactome collections we found that cytokine-dependent mechanisms and NK-mediated cytotoxicity represent enriched mechanisms. These findings are consistent with previous studies in the immunobiology of MM, however a detailed profile of the surface proteins mediating these mechanisms had remained unknown or only partially known so-far.

### Normal Tissue Annotation Identifies Targetable Antigens

In order to identify therapeutically relevant targets, we used a pipeline we previously established for leukemia. This combines three public proteomic databases for human proteome annotation (Human Protein Atlas, Human Protein Map and Proteomics DataBase) and allowed the annotation of each candidate protein in a panel of 42 normal tissues and organs. Given that, we excluded proteins with high expression in any normal tissue except hematopoietic tissues and with an available annotation in less than two out of the three databases. Through this, we identified 94 out of 326 targets with minimal expression in normal tissues. This list includes BCMA, SLAMF7, ITGB7, TACI and Ly9. We also ranked each one of the 94 targets based on their expression in normal tissues and thus, predicted on-target off-tumor toxicity.

By overlapping the list of immune-related proteins (227) with that of proteins with a favorable profile in normal tissues we obtained 67 targets with both functional and therapeutic relevance (FIG. 3). Such latter list still includes BCMA, CD229, SLAMF7, ITGB7, TACI that are targeted in current pre-clinical trials for immunotherapy of MM.

### Validation in Primary Patient Samples Identifies 11 Targets

Based on the expression in normal tissues we chose 24 targets for further validation in primary MM patient samples. Eleven of these targets include CCR1 (SEQ ID NO: 60), CD320 (SEQ ID NO: 56), FCRL3(SEQ ID NO: 57), IFNGR1 (SEQ ID NO: 79), IL12RB1 (SEQ ID NO: 19), ITGA4 (SEQ ID NO: 42), LILRB4 (SEQ ID NO: 20), LRRC8D (SEQ ID NO: 112), SEMA4A (SEQ ID NO: 104), and SLAMF6 (SEQ ID NO: 37) and BCMA, and in some aspects, these targets can be integrated into modified or synthesized chimeric antigen receptors, antibodies or antibody binding fragments thereof, and immune cells including T-Cells containing or expressing the foregoing.

1. A method for identifying target multiple myeloma associated surface antigens, said method comprising:
identifying a plurality of genes that express cell-surface proteins in a first multiple myeloma sample and a second multiple myeloma sample;
selecting nucleic acids from said first multiple myeloma sample that have expression levels higher than a control gene unrelated to hematopoietic cells, and identifying the proteins corresponding to the detected elevated expressed nucleic acids to designate a first pool of selected proteins;
conducting mass spec analysis on proteins isolated from said second myeloma sample to identify proteins that are present in higher concentration in said second multiple myeloma relative to normal tissues, wherein such proteins represent a second pool of selected proteins;
excluding proteins with high expression in brain, spinal cord, gut, liver and kidney from said first and second pools to produce a modified first and second pool of proteins; and
identifying proteins common to said first and second modified pool of proteins as target multiple myeloma associated surface antigens.

2. The method of claim 1 wherein said first multiple myeloma sample and said second multiple myeloma sample are taken from the same tissue source.

3. The method of claim 1 wherein said first multiple myeloma sample is a nucleic acid pool of expressed genes from MM patients and said second multiple myeloma sample represents proteins expressed in MM cell lines.

4. The method of claim 1, wherein the target multiple myeloma associated surface antigen has an expression level in a normal tissue sample that is more than about one standard deviation below the normal peak of the protein expression level distribution of the normal tissue sample.

5. The method of claim 1, wherein mRNA is measured to determine the expression level of the nucleic acids used to identify proteins for the first pool of selected proteins.

6. A monoclonal antibody that specifically binds to
i) a polypeptide having at least 90% sequence identity to a polypeptide selected from the group consisting of SEQ ID NO: 1-155 or 168-208; or
ii) a polypeptide selected from the group consisting of SEQ ID NO: 19, SEQ ID NO: 20, SEQ ID NO: 37, SEQ ID NO: 42, SEQ ID NO: 54, SEQ ID NO: 56, SEQ ID NO: 57, SEQ ID NO: 79, SEQ ID NO: 112 and SEQ ID NO: 104; or
iii) a polypeptide having at least 90% sequence identity to a sequence selected from the group consisting of CCR1 (SEQ ID NO: 60), CD320 (SEQ ID NO: 56), FCRL3(SEQ ID NO: 57), IFNGR1 (SEQ ID NO: 79), IL12RB1 (SEQ ID NO: 19), ITGA4 (SEQ ID NO: 42), LILRB4 (SEQ ID NO: 20), LRRC8D (SEQ ID NO: 112), SEMA4A (SEQ ID NO: 104), and SLAMF6 (SEQ ID NO: 37); or
iv) to a polypeptide having at least 90% sequence identity to a sequence selected from the group consisting of SEQ ID NO: 1-155 or 168-208.

7-11. (canceled)

12. The monoclonal antibody of claim 6 further comprising a detectable label covalently linked to the antibody or a cytotoxic agent linked to the antibody.

13. (canceled)

14. A chimeric antigen receptor (CAR) comprising
an antibody, or antigen binding fragment thereof, that binds one or more epitopes of a polypeptide selected from the group consisting of SEQ ID NO: 1-155 or 168-208,
a transmembrane domain; and
an immune cell antigen receptor chain, wherein the transmembrane domain links the an antibody, or antigen binding fragment thereof to the immune cell antigen receptor chain.

15. The chimeric antigen receptor of claim 14 wherein the antibody or antigen binding fragment thereof, specifically binds to
i) a polypeptide selected from the group consisting of SEQ ID NO: 168-208; or
ii) a polypeptide having at least 95% sequence identity to a sequence selected from the group consisting of CCR1 (SEQ ID NO: 60), CD320 (SEQ ID NO: 56), FCRL3(SEQ ID NO: 57), IFNGR1 (SEQ ID NO: 79), IL12RB1 (SEQ ID NO: 19), ITGA4 (SEQ ID NO: 42), LILRB4 (SEQ ID NO: 20), LRRC8D (SEQ ID NO: 112), SEMA4A (SEQ ID NO: 104) and SLAMF6 (SEQ ID NO: 37);
iii) one or more epitopes of a polypeptide having at least 90% homology to a sequence selected from the group consisting of SEQ ID NO: 1-155 or 168-208
iv) binds to a polypeptide selected from the group consisting of SEQ ID NO: 19, SEQ ID NO: 20, SEQ ID NO: 37, SEQ ID NO: 42, SEQ ID NO: 54, SEQ ID NO: 56, SEQ ID NO: 57, SEQ ID NO: 79, SEQ ID NO: 112 and SEQ ID NO: 104.

16-19. (canceled)

20. The chimeric antigen receptor of claim 15 wherein said antibody or antigen binding fragment comprises an antibody single-chain variable fragment, optionally further comprising a hinge region located between the antibody single-chain variable fragment and the transmembrane domain.

21. (canceled)

22. A T-cell modified to express the chimeric antigen receptor of claim 20, optionally wherein the T-cell is a tumor infiltrating leukocyte.

23. (canceled)

24. A cell modified to express the chimeric antigen receptor of claim 20, wherein the cell is selected from the group consisting of an NK, macrophage and myeloid cell.

25. (canceled)

26. (canceled)

27. The T-cell of claim 22 wherein the T-cell antigen receptor chain is the CD3ζ chain (zeta-chain).

28. A pharmaceutical composition comprising the antibody of claim 6.

29. A method for treating multiple myeloma, said method comprising administering the pharmaceutical composition of claim 28 to a patient in need of such treatment.

30. An isolated immunoresponsive cell comprising an antigen recognizing receptor that binds to an antigen selected from the group consisting of CCR1 (SEQ ID NO: 60), CD320 (SEQ ID NO: 56), FCRL3(SEQ ID NO: 57), IFNGR1 (SEQ ID NO: 79), IL12RB1 (SEQ ID NO: 19), ITGA4 (SEQ ID NO: 42), LILRB4 (SEQ ID NO: 20), LRRC8D (SEQ ID NO: 112), SEMA4A (SEQ ID NO: 104), and SLAMF6 (SEQ ID NO: 37).

31. The isolated immunoresponsive cell of claim 30, wherein the antigen is selected from the group consisting of IL12RB1 (SEQ ID NO: 19), LILRB4 (SEQ ID NO: 20), SLAMF6 (SEQ ID NO: 37), CCR1 (SEQ ID NO: 60), and CD320 (SEQ ID NO: 56).

32. The isolated immunoresponsive cell of claim 31, wherein said antigen recognizing receptor is a T cell receptor (TCR), or a chimeric antigen receptor (CAR).

33. (canceled)

34. The isolated immunoresponsive cell of claim 33, wherein the intracellular signaling domain of said CAR is the CD3C-chain, CD97, CD1 la-CD 18, CD2, ICOS, CD27, CD 154, CD8, OX40, 4-IBB, CD28 signaling domain, or combinations thereof.

35. The isolated immunoresponsive cell of claim 34 wherein the cell is selected from the group consisting of a T cell, a Natural Killer (NK) cell, a cytotoxic T lymphocyte (CTL), a regulatory T cell, a Natural Killer T (NKT) cell, a human embryonic stem cell, and a pluripotent stem cell from which lymphoid cells may be differentiated.

36. An isolated immunoresponsive cell of claim 30 wherein said immunoresponsive cell comprises:
(a) a first antigen recognizing receptor that binds to a first antigen, and
(b) a second antigen recognizing receptor that binds to a second antigen, wherein each of the first antigen and the second antigen is selected from the group consisting of CCR1 (SEQ ID NO: 60), CD320 (SEQ ID NO: 56), FCRL3(SEQ ID NO: 57), IFNGR1 (SEQ ID NO: 79), IL12RB1 (SEQ ID NO: 19), ITGA4 (SEQ ID NO: 42), LILRB4 (SEQ ID NO: 20), LRRC8D (SEQ ID NO: 112), SEMA4A (SEQ ID NO: 104), and SLAMF6 (SEQ ID NO: 37), and the first antigen and the second antigen are different.

37. The isolated immunoresponsive cell of claim 36, wherein each of said antigen recognizing receptor is a T cell receptor (TCR) or a chimeric antigen receptor (CAR).

38. (canceled)

39. A method of reducing tumor burden in a subject, comprising administering to the subject an effective amount of the immunoresponsive cell of claim 36.

40. A pharmaceutical composition comprising the T-cell of claim 22.
